(19) United States
(12) Patent Application Publication
(10) Pub. No.: US 2005/0244964 A1
(43) Pub. Date: Nov. 3, 2005

(54) Production of neural progenitor cells

(76) Inventor: Bruce Paul Davidson, Helios (SG)

Correspondence Address: Sutherland, Asbill & Brennan/Atta: Bill Warren, 999 Peachtree Street, NE, Atlanta, GA, 30309-3996, US

(21) Appl. No.: 10/514,094

(22) PCT Filed: May 9, 2003
(86) PCT No.: PCT/AU03/00552

§ 371 Date: Jun. 27, 2005

(30) Foreign Application Priority Data
May 10, 2002 (AU) PS 2254

(51) Int. Cl. C12N 5/08

(52) U.S. Cl. 435/368

(57) ABSTRACT

A method of producing neural progenitor cells and/or neuronal cells which method includes providing a source of pluripotent cells; a cell aggregate-inducing culture medium; and a neural inducing supplement; culturing the pluripotent cells in the cell aggregate-inducing culture medium, in the presence of the neural inducing supplement, for a period sufficient to permit cell aggregates or embryoid bodies (EB's) to form, wherein the EB's include neural progenitor cells; and culturing the cell aggregates including neural progenitor cells for a period sufficient to permit neuronal differentiation.

The present invention relates to improved methods of producing, differentiating and culturing neuronal and neural precursor cells, to methods of producing neurospheres, and to uses thereof.

The present invention further relates to methods for producing cell populations including a high proportion of tyrosine hydroxylase positive (TH+ve) cells, more particularly cell populations of differentiated neuronal cells including a high proportion of TH+ve neuronal cells.

Initial developmental events within the mammalian embryo entail the elaboration of extra-embryonic cell lineages and result in the formation of the blastocyst, which comprises trophectoderm, primitive endoderm and a pool of pluripotent cells, the inner cell mass (ICM/epiblast). As development continues, the cells of the ICM/epiblast undergo rapid proliferation, selective apoptosis, differentiation and reorganisation as they develop to form the primitive ectoderm. In the mouse, the cells of the ICM begin to proliferate rapidly around the time of blastocyst implantation. The resulting pluripotent cell mass expands into the blastocoelic cavity. Between 5.0 and 5.5 dpc (days post coitus) the inner cells of the epiblast undergo apoptosis to form the proamniotic cavity. The outer, surviving cells, or early primitive ectoderm, continue to proliferate and by 6.0 to 6.5 dpc have formed a pseudo-stratified epithelial layer of pluripotent cells, termed the primitive or embryonic ectoderm. Primitive ectoderm cells are pluripotent, and distinct from cells of the ICM in terms of morphology, gene expression and differentiation potential.

By 4.5 dpc pluripotent cells exposed to the blastocoelic cavity have differentiated to form primitive endoderm. The primitive endoderm gives rise to two distinct endodermal cell populations, visceral endoderm, which remains in contact with the epiblast, and parietal endoderm, which migrates away from the pluripotent cells to form a layer of endoderm adjacent to the trophectoderm. Formation of these endodermal layers is coincident with formation of primitive ectoderm and creation of an inner cavity. Visceral endoderm is known to express signals that influence pluripotent cell differentiation.

At gastrulation pluripotent cells of the primitive ectoderm differentiate to form the three germ layers of the embryo: mesoderm, endoderm and ectoderm. Pluripotent cells from this time are confined to the germline. Differentiation of primitive ectoderm cells in the distal and anterior regions of the embryo is directed along the ectodermal lineage forming definitive ectoderm, a transient embryonic cell type fated to form neurectoderm and surface ectoderm.

Neurectoderm cells are found in the mammalian embryo in the neural plate, which folds and closes to form the neural tube. These cells are the precursors to all neural lineages. They have the capacity to differentiate into all neural cell types present in the central nervous system (CNS) and peripheral nervous system (PNS). In the CNS these cells include multiple neuron subtypes and glia (eg; astrocytes and oligodendrocytes). Neural cells of the peripheral nervous system also include many different types of neurons and glial cells. Peripheral neural cells differentiate from transient embryonic precursor cells termed neural crest cells, which arise from the neural tube. Neural crest cells are also precursor cells to non-neural cells, including melanocytes, cartilage and connective tissue of the head and neck, and cells of cardiac outflow septation (Anderson, 1989).

In the human and in other mammals, formation of the blastocyst, including development of ICM cells and their progression to pluripotent cells of the primitive ectoderm, and subsequent differentiation to form the embryonic germ layers and differentiated cells, follow a similar developmental process.

Pluripotent cells can be isolated from the preimplantation mouse and human embryos as embryonic stem (ES) cells. ES cells can be maintained indefinitely as a pluripotent cell population in vitro. When reintroduced into a host murine blastocyst, mouse ES cells can contribute to all adult tissues of the mouse including the germ cells. ES cells, therefore, retain the ability to respond to all the signals that regulate normal mouse development. EPL cells are a separate population of pluripotent cells distinct from ES cells. EPL cells are equivalent to early primitive ectoderm cells of the post-implantation embryo, and can be maintained, proliferated and differentiated in a controlled manner in vitro. EPL cells and their properties are described in International patent application WO99/53021, to applicants.

ES cells and EPL cells represent powerful model systems for the investigation of mechanisms underlying pluripotent cell biology and differentiation within the early embryo, as well as providing opportunities for embryo manipulation and resultant commercial, medical and agricultural applications. Furthermore, appropriate proliferation and differentiation of ES and EPL cells can be used to generate an unlimited source of cells suited to transplantation for treatment of diseases which result from cell damage or dysfunction.

Other pluripotent cells and cell lines including in vivo or in vitro derived ICM/epiblast, in vivo or in vitro derived primitive ectoderm, primordial germ cells (EG cells), teratocarcinoma cells (EC cells), and pluripotent cells derived by dedifferentiation or by nuclear transfer will share some or all of these properties and applications.

The successful isolation, long term clonal maintenance, genetic manipulation and germ-line transmission of pluripotent cells from species other than rodents has generally been difficult to date and the reasons for this are unknown. International patent application WO97/32033 and U.S. Pat. No. 5,453,357 describe pluripotent cells including cells from species other than rodents. Primate ES cells have been described in International patent application WO96/23362, and in U.S. Pat. No. 5,843,780, and human EG cells have been described in International patent application WO98/43679.

The differentiation of murine ES cells can be regulated in vitro by the cytokine leukaemia inhibitory factor (LIF) and other gp130 agonists or by culture on feeder cells which promote self-renewal and prevent differentiation of the stem cells. Differentiation in vitro of human ES cells is not inhibited by LIF, but is inhibited by culture on feeder cells.

The ability to form predominantly homogeneous populations of partially differentiated or terminally differentiated cells by differentiation in vitro of pluripotent cells has proved problematic. Current approaches involve the formation of embryoid bodies from pluripotent cells, in a manner that is not controlled and does not result in homogeneous populations. Mixed cell populations such as those in embryoid bodies of this type are generally unlikely to be suitable for therapeutic or commercial use.

Selection procedures have been used to obtain cell populations enriched in neural cells from embryoid bodies. These include manipulation of culture conditions to select for neural cells (Okabe et al, 1996), and genetic modification of ES cells to allow selection of neural cells by antibiotic resistance (Li et al, 1998). Neurospheres presumably comprising neural precursors have also been produced with low efficiency (Tropepe et al, 2001).

In these procedures the differentiation of pluripotent cells in vitro does not direct differentiation in a controlled manner. Hence homogeneous synchronous, populations of neurectoderm cells with specific neural differentiation capability are not produced, constraining the ability to derive essentially homogeneous populations of partially differentiated or differentiated neural cells.

Chemical inducers such as retinoic acid have also been used to form neural lineages from a variety of pluripotent cells including ES cells (Bain et al, 1995). However the route of retinoic acid-induced neural differentiation has not been well characterised, and the repertoire of neural cell types produced appears to be generally restricted to ventral somatic motor, branchiomotor or visceromotor neurons (Renoncourt et al, 1998).

In summary it has not been possible to control the differentiation of pluripotent cells in vitro, to provide homogeneous, synchronous populations of neurectoderm cells with unrestricted neural differentiation capacity. Similarly methods have not been developed for the derivation of neurectoderm cells from pluripotent cells, in a manner that parallels their formation during embryogenesis. These limitations have restricted the ability to form essentially homogeneous, synchronous populations of partially differentiated and terminally differentiated neural cells in vitro, and have restricted their further development for therapeutic and commercial applications.

Neural stem cells and precursor cells have also been derived from foetal brain and adult primary central nervous system tissue in a number of species, including rodent and human (e.g. see U.S. Pat. No. 5,753,506 (Johe), U.S. Pat. No. 5,766,948 (Gage), U.S. Pat. No. 5,589,376 (Anderson and Stemple), U.S. Pat. No. 5,851,832 (Weiss et al), U.S. Pat. No. 5,958,767 (Snyder et al) and U.S. Pat. No. 5,968,829 (Carpenter). However, each of these disclosures fails to describe a predominantly homogeneous population of neural stem cells able to differentiate into all neural cell types of the central and peripheral nervous systems, and/or essentially homogeneous populations of partially differentiated or terminally differentiated neural cells derived from neural stem cells by controlled differentiation.

Furthermore, it is not clear whether cells derived from primary foetal or adult tissue can be expanded sufficiently to meet potential cell and gene therapy demands.

International patent application WO 01/51611 to applicants, describes the production of neurectoderm cells from EPL cells utilising a specific conditioned medium. Whilst this is a significant advance, the cell lines produced may be sub-optimal where the cells are not sufficiently committed to the neural lineage and teratomas may be formed.

It is an object of the present invention to overcome, or at least alleviate, one or more of the difficulties or deficiencies associated with the prior art.

In a first aspect of the present invention, applicants have discovered that it is possible to generate neuronal or neural progenitor cells from pluripotent cells, e.g. cell aggregates (or embryoid bodies).

Accordingly, there is provided a method of producing neural progenitor cells and/or neuronal cells which method includes
providing
   a source of pluripotent cells;
   a cell aggregate-inducing culture medium; and
   a neural inducing supplement;
culturing the pluripotent cells in the cell aggregate-inducing culture medium, in the presence of the neural inducing supplement, for a period sufficient to permit cell aggregates or embryoid bodies (EB's) to form, wherein the EB's include neural progenitor cells; and
culturing the cell aggregates including neural progenitor cells for a period sufficient to permit neuronal differentiation.

Applicants have found that by generating neural and neuronal progenitor cells, a cell population is provided that may be useful in treating neural diseases when transplanted into an animal subject. Applicants have also found that such a cell population is also useful for generating neurospheres, which are cell populations highly enriched in neural precursors. Neurosphere cells, and neural cells derived from neurospheres may also be useful in treating neural diseases when transplanted into an animal subject.

The cell aggregate-inducing culture medium may be any suitable culture medium which will permit the production and growth of cell aggregates, in particular those containing neuronal or neural progenitor cells. It is particularly preferred that the pluripotent cells are aggregated in a culture medium such as Dulbecco's Modified Eagles Medium (DMEM), supplemented with a neural inducing supplement.

Preferably the neural inducing supplement is a hormone or growth supporting supplement. More preferably the neural inducing supplement is ITSS and/or B27 and/or N2.

Desirably, the culture medium is serum-free, that is it excludes foetal cell serum (FCS) or the like.

Whilst the cell aggregate-inducing medium supplemented with the neural inducing supplement may include a fibroblast growth factor, eg. FGF-2, it is preferred that the cell culturing steps are conducted in the absence of a fibroblast growth factor.

In a preferred aspect of the present invention the cell aggregate-inducing medium further includes retinoic acid, an isomer thereof, precursor thereof or derivative thereof.

The retinoic acid source, when present, may be of any suitable type. Retinoic acid (RA), (all-E)-3,7-dimethyl-9-(2,6,6-trimethyl-1-cyclohexen-1-yl)-2,4,6,8-nonatetraenoic acid is a physiological metabolite of retinol, a compound of vitamin A. An acid or salt may be used. A retinoic acid isomer or mixture of isomers may be used. A retinoic acid precursor such as retinaldehyde may be used. The all-trans retinoic acid isomer is preferred.

The cell aggregates or embryoid bodies may be generated in adherent culture or in suspension culture. It is particularly preferred that the pluripotent cells are aggregated in suspension culture.

Preferably the cells are initially cultured for approximately 3 to 10 days, more preferably approximately 4 to 9 days, most preferably approximately 9 days. Typically the cell aggregates at approximately 9 days are enriched in neural precursor cells that resemble neurectoderm cells as described in International application WO 01/5161 1, the entire disclosure of which is incorporated herein by reference. Embryoid bodies so produced at approximately 9 days may also include partially differentiated and terminally differentiated neuronal cells, oligodendrocytes and glia.

As used herein, the term “neurectoderm” refers to undifferentiated neural progenitor cells substantially equivalent to cell populations comprising the neural plate and/or neural tube. Neurectoderm cells referred to herein potentially retain the capacity to differentiate into all neural lineages, including neurons, oligodendrocytes and glia of the central nervous system, and neural crest cells able to form all cell types of the peripheral nervous system.

In a preferred aspect of the present invention embryoid bodies, for example, at approximately 9 days, are cultured for an additional period of time so that neuronal differentiation may proceed. The additional period of time is preferably approximately 4 to 20 days, and more preferably approximately 6 to 9 days.

In a preferred aspect of the present invention the additional culture is conducted in a medium that is the same as the cell aggregate-inducing medium and preferably includes neural inducing supplement.

In a further preferred aspect of the present invention, the cell aggregation is conducted in a suspension culture and the additional culture, during which further neuronal differentiation occurs, is conducted in a suspension or adhesion culture. Most preferably the additional neuronal differentiation culture is conducted in adherent culture.

The pluripotent cells may be selected from one or more of the group consisting of embryonic stem (ES) cells, early primitive ectoderm-like (EPL) cells, in vivo or in vitro derived ICM/epiblast, in vivo or in vitro derived primitive ectoderm, primordial germ cells (EG cells), teratocarcinoma cells (EC cells), and pluripotent cells derived by dedifferentiation or by nuclear transfer. EPL cells may also be derived from differentiated cells by dedifferentiation. The pluripotent cells may be of animal, particularly mammalian origin. The pluripotent cells may be of human or murine origin.

Cells derived from ES cells or EPL cells are preferred.

Where embryonic stem (ES) cells are used, they are preferably ES cells which are positive to a cell surface marker, eg. SSEA4. Thus, the desired ES cells may be selected utilising an anti-SSEA4 antibody.

The SSEA4+ve ES cells may be proliferated in culture, preferably in the presence of a fibroblast growth factor, eg. FGF-2.

Applicants have surprisingly found that the SSEA4+ve ES cells may express nestin, an early neural cell marker, whilst retaining the ability to express standard pluripotent cell markers, as discussed above.

It is preferred that cell-to-cell contact of pluripotent cells is disrupted, for example by trituration or enzyme digestion such as trypsinisation, prior to initiation of aggregation culturing.

In a preferred embodiment of the invention there is provided a method of producing tyrosine hydroxylase positive cells which method includes
providing
   a source of pluripotent cells;
   a cell aggregate-inducing culture medium;
   a neural inducing supplement; and
   a tyrosine hydroxylase (TH)-inducing supplement;
culturing the pluripotent cells in the cell aggregate-inducing culture medium, in the presence of the neural inducing supplement and TH-inducing supplement, for a period sufficient to permit cell aggregates or embryoid bodies (EB's) to form, wherein the EB's include neural progenitor cells; and
culturing the cell aggregates including neural progenitor cells for a period sufficient to permit differentiation of neuronal cells, wherein said neuronal cells express tyrosine hydroxylase.

Applicants have surprisingly found that cells grown in the presence of a neural-inducing supplement and TH-inducing supplement may include a high proportion of neuronal cells, wherein the neuronal cells may be largely comprised of tyrosine hydroxylase positive (TH+ve) cells, eg. at least approximately 5% up to approximately 50% or greater of TH+ve cells. Neuronal cells that are TH+ve may exhibit dopaminergic characteristics in vivo.

Such dopaminergic neuronal cells may be suitable for alleviating symptoms of Parkinson's disease when implanted into an animal subject exhibiting symptoms of Parkinson's disease.

The TH-inducing supplement utilised in the method according to the present invention may include a conditioned medium, or filtrate fraction thereof, as described in International patent application WO99/53021, the entire disclosure of which is incorporated herein by reference.

The term “conditioned medium” includes within its scope a filtrate fraction thereof including medium components below approximately 10 kDa, and/or a fraction thereof including medium components above approximately 10 kDa.

Most preferably when a conditioned medium is used as the TH-inducing supplement, the fraction thereof that includes medium components below approximately 10 kDa is used.

Preferably the conditioned medium is prepared using a hepatic or hepatoma cell or cell line, more preferably a human hepatocellular carcinoma cell line such as Hep G2 cells (ATCC HB-8065) or Hepa-1c1c-7 cells (ATCC CRL-2026), primary embryonic mouse liver cells, primary adult mouse liver cells, or primary chicken liver cells, or an extraembryonic endodermal cell or cell line such as the cell lines END-2 and PYS-2. However, the conditioned medium may be prepared from a medium conditioned by liver or other cells from any appropriate species, preferably mammalian or avian. The conditioned medium MEDII, as descried in WO99/53021, is particularly preferred.

A TH-inducing extract from the conditioned medium may be used in place of the conditioned medium. Optionally, the TH-inducing extract does not include the biologically active factor, conditioned medium or the large or low molecular weight component thereof. The term “TH-inducing extract” as used herein includes within its scope a natural or synthetic molecule or molecules which exhibit(s) similar biological activity, e.g. a molecule or molecules which compete with molecules within the conditioned medium that bind to a receptor on EPL cells responsible for neural induction.

As described in International patent application WO99/53021, the conditioned medium or filtrate fraction thereof includes proline, and/or proline-containing peptides. Accordingly, in a preferred form of the invention the TH-inducing agent used to supplement the cell aggregate-inducing medium may include a source of proline, preferably at a concentration of 50 μM or greater, most preferably at a concentration of 100 μM.

More preferably the cell aggregate-inducing medium is supplemented with a neural inducing supplement as herein before described, and a TH-inducing supplement in the form of nutrient media such as a Ham's F12 nutrient medium as a source of proline. Most preferably the cell aggregate-inducing medium is a Dulbecco's Modified Eagles Medium (DMEM), supplemented with a neural inducing supplement as herein before described, and Ham's F12 nutrient media.

Desirably, the culture medium is serum-free, that is it excludes foetal cell serum (FCS) or the like.

Whilst the cell aggregate-inducing medium supplemented with the neural inducing supplement and TH-inducing supplement may include a fibroblast growth factor, eg. FGF-2, it is preferred that the cell culturing steps are conducted in the absence of a fibroblast growth factor.

In a preferred aspect of the present invention the cell aggregate-inducing medium further includes retinoic acid, an isomer thereof, precursor thereof or derivative thereof.

The cell aggregates or embryoid bodies may be generated in adherent culture or in suspension culture. It is particularly preferred that the pluripotent cells are aggregated in suspension culture.

Preferably the cells are initially cultured for approximately 3 to 10 days, more preferably approximately 4 to 9 days, most preferably approximately 9 days.

In a preferred aspect of the present invention embryoid bodies at approximately 9 days are cultured for an additional period of time so that differentiation to TH+ve neuronal cells may proceed. The additional period of time may be approximately 4 to 20 days, and most preferably approximately 6 to 9 days.

In a preferred aspect of the present invention the additional culture is conducted in a medium that is the same as the cell aggregate-inducing medium and preferably includes neural inducing supplement and TH-inducing supplement.

In a further preferred aspect of the present invention, the cell aggregation is conducted in a suspension culture and the additional culture, during which further differentiation to TH+ve neuronal cells occurs, is conducted in a suspension or adhesion culture. Most preferably the additional culture is conducted in adherent culture.

The cell aggregates so formed include at least approximately 5% neuronal cells, more preferably approximately 50% neuronal cells. It is particularly preferred that at least approximately 5%, more preferably at least approximately 50% of the cells are TH+ve, ie, dopaminergic.

In a preferred aspect of the present invention, there is provided a method of producing neurospheres, which method includes
providing
   a source of pluripotent cells;
   a cell aggregate-inducing culture medium;
   a neural inducing supplement;
   optionally a TH-inducing supplement; and
   a neurosphere-inducing culture medium;
culturing the pluripotent cells in the cell aggregate-inducing culture medium, in the presence of the neural inducing supplement and optionally in the presence of the TH-inducing supplement for a period sufficient to permit cell aggregates or embryoid bodies (EB's) to form;
disaggregating the embryoid bodies;
culturing the cells so released in the neurosphere-inducing culture medium to form neurospheres; and
harvesting the neurospheres so formed.

Applicants have found that by generating neural progenitor cells as neurospheres, as hereinafter described, difficulties with the production of tumors, including teratomas, in vivo when neural cells are transplanted into an animal subject, may be reduced or eliminated.

Neurospheres are self-adherent clusters of multipotent neural cells which may be formed under specific culture conditions.

Preferably the cell aggregate-inducing medium is a Dulbecco's Modified Eagles Medium (DMEM) supplemented with a neural inducing supplement, eg. ITSS, B27 and/or N2. Optionally a TH-inducing supplement is included in the cell aggregate-inducing medium.

Whilst the cell aggregate-inducing medium may include a fibroblast growth factor, eg, FGF-2, it is preferred that the cell aggregation steps are conducted in the absence of a fibroblast growth factor.

Preferably the neurosphere-inducing culture medium includes a serum-free medium, more preferably a serum-free Dulbecco's Modified Eagles Medium (DMEM). The culture medium may be further supplemented with additional growth factors, including a growth factor from the FGF family (eg. FGF-2) and/or differentiation agents and/or growth additives, eg. selected from one or more of the group consisting of heparin (e.g. at approximately 10 μg/ml), B27 and ITSS. Optionally the neurosphere-inducing culture medium is further supplemented with a conditioned medium such as MEDII or extract thereof, or a source of proline such as a Ham's F12 nutrient medium.

Neurospheres may be formed from embryoid bodies cultured in aggregation inducing medium for approximately 6 to 25 days, more preferably for approximately 9 to 18 days. Inclusion of retinoic acid in the cell aggregation medium may result in early embryoid bodies that are able to produce neurospheres.

Embryoid bodies are dissociated to single cells or near single cells by enzymatic treatment or by physical means such as trituration. The cells are cultured in neurosphere-inducing culture in suspension culture or adherent culture. Preferably the culture is in suspension.

Neurospheres may begin to appear in a time-frame of approximately 3 to 9 days, preferably approximately 4 to 8 days, after neurosphere culturing is initiated.

Accordingly in a preferred aspect of this embodiment of the present invention, the neurospheres may be passaged and grown in serum-free culture medium to yield tertiary spheres prior to the harvesting of the neuronal and/or neural progenitor cells.

In a preferred aspect, the method further includes
maintaining the neurospheres in a serum-free culture media prior to harvesting, eg. for approximately 1 to 21 days.

In a still further aspect of the present invention, there is provided a method of producing neuronal and/or neural progenitor cells which method includes
providing
   a source of neurospheres; and
   a neuronal differentiation culture medium; and
   culturing the neurospheres in the presence of the neural differentiation medium for a period sufficient to permit neuronal differentiation.

The neurospheres may be produced as described above.

The neuronal differentiation medium is preferably a Dulbecco's Modified Eagles Medium (DMEM), preferably in the absence of a fibroblast growth factor. Optionally the neuronal differentiation medium is supplemented with a conditioned medium as herein before described, or a source of proline as herein before described.

Accordingly, the present invention further provides differentiated neuronal cells produced by the method as described above. Preferably approximately 5% to approximately 50% of the cells are neuronal cells. More preferably approximately 5% to approximately 50% of the cells are tyrosine hydroxylase positive (TH+ve).

The neurospheres formed according to this aspect of the present invention may be characterised in that they may produce cells of all three neuronal lineages and with a reduced propensity to generate teratomas in vivo.

Accordingly, in this aspect of the present invention, there is further provided neurospheres produced by the method described above and capable of producing cells of all three neuronal lineages, or the partially or terminally differentiated progeny thereof.

The neurospheres may be of mammalian, including human, origin.

The neurospheres may be further characterised in that
proliferating cells are present (cells positive to Ki67 marker) and
neuronal cells are present (cells positive to NF200 marker).

The neurospheres may be further characterised in that
a proportion of cells, preferably approximately 50% or greater, are dopaminergic (cells positive to Tyrosine hydroxylase (TH) marker).

The neurospheres may further include glial cells (cells positive to GFAP marker).

In a further aspect of the present invention, neurospheres and differentiated progeny of the neurosphere cells have a reduced propensity to generate teratomas in vivo when passaged in a serum-free medium.

Accordingly, in this aspect, the method of producing neurospheres further includes subsequently maintaining the neurospheres in a serum-free culture media prior to harvesting.

The neurospheres may be in the serum-free culture media for approximately 1 to 40 days, preferably approximately 1 to 21 days.

The neurospheres or neuronal cells of the present invention and the differentiated or partially differentiated cells derived therefrom are well defined, and can be generated in amounts that allow widespread availability for therapeutic and commercial uses. The cells have a number of uses, including the following:

Use in human cell therapy to treat and cure neurodegenerative disorders such as Parkinson's disease, Huntington's disease, lysosomal storage diseases including (α-Mannosidosis, multiple sclerosis, memory and behavioural disorders, Alzheimer's disease and macular degeneration, and other pathological conditions including stroke and spinal chord injury. For example genetically modified or unmodified neurospheres, or their differentiated or partially differentiated progeny may be used to replace or assist the normal function of diseased or damaged tissue.

Further, for example in Parkinson's disease, the dopaminergic cells of the substantia nigra are progressively lost. The dopaminergic cells in Parkinson's patients may be replaced by implantation of neural cells produced in the manner described in this application.

In a still further example, a-Mannosidosis is a lysosomal storage disorder (LSD) caused by a genetic deficiency of the lysosomal enzyme α-mannosidase, and is characterised primarily by progressive neurological degeneration in the central nervous system (CNS). Initial animal studies involve injection of neural cell progenitors, neurospheres or the progeny thereof, into the striatum or brains of normal guinea pigs.

Use to produce cells, tissues or components of organs for transplant. For example neural crest cells retain the capacity to form non-neural cells, including cartilage and connective tissue of the head and neck, and are potentially useful in providing tissue for craniofacial reconstruction.

Use in human gene therapy to treat neuronal and other diseases. In one approach neurospheres or their differentiated and partially differentiated products may be genetically modified; eg; so that they provide functional biological molecules. The genetically modified cells can be implanted, thus allowing appropriate delivery of therapeutically active molecules.

Use as a source of cells for reprogramming. For example karyoplasts from neurospheres or their differentiated or partially differentiated progeny may be reprogrammed by nuclear transfer. Cytoplasts from neurectodermal cells may also be used as vehicles for reprogramming so that nuclear material derived from other cell types are directed along neural lineages. Alternatively neural stem cells may be reprogrammed in response to environmental and biological signals to which they are not normally exposed.

For example, the differentiation of murine neural stem cells is redirected to form haematopoietic cells (cells of mesodermal lineage), when injected into the bone marrow (eg; Bjornson et al, 1999). Hence neural progenitor cells described herein are potentially capable of forming differentiated cells of non-neural lineages, including cells of mesodermal lineage, such as haematopoietic cells and muscle. Reprogramming technology using neural cells potentially offers a range of approaches to derive cells for autologous transplant. In one approach karyoplasts from differentiated cells are obtained from the patient, and reprogrammed in neural progenitor cytoplasts to generate autologous neural progenitors.

The autologous neurospheres, or their differentiated or partially differentiated progeny may then be used in cell therapy to treat neurodegenerative diseases.

Use in pharmaceutical screening for therapeutic drugs that influence the behaviour of neurospheres, and their differentiated or partially differentiated progeny. Neurosphere cells may be particularly appropriate in evaluating the toxicology and teratogenetic properties of pharmaceutically useful drugs, since many birth defects, including spina bifida are caused by failures in neural tube closure.

Use in the identification and evaluation of biological molecules that direct differentiation of neural cells or neural precursors, including patterning molecules.

Use in identifying genes expressed in neurosphere, cells and partially differentiated or differentiated neural cells.

Accordingly, in a further aspect of the present invention, there is provided a method for the treatment of neuronal and other diseases, as described above, which method includes treating a patient requiring such treatment with genetically modified or unmodified neurospheres or neuronal or neural progenitor cells as described above, or their partially differentiated or terminally differentiated progeny, through human or animal cell or gene therapy.

In a still further aspect of the present invention, there is provided a method for the preparation of tissue or organs for transplant, which method includes
providing neural crest cells or neurectoderm produced as described above; and
culturing the neural crest cells to produce neural or non-neural cells and the neurectoderm cells to produce neural cells.

The present invention will now be more fully described with reference to the accompanying examples and drawings. It should be understood, however, that the description following is illustrative only and should not be taken in any way as a restriction on the generality of the invention described above.

### IN THE FIGURES

FIG. 1 shows examples of embryoid bodies grown in either (A) 50% Medll or (B) 50% MEDII supplemented with 100 nM all-trans Retinoic Acid. Note that embryoid bodies grown in 50% MEDII without RA supplementation exhibit regions with epithelial morphology as well as less structured cell types whereas supplementation with RA increases the degree and uniformity of epithelial tissue (neurectoderm) present. Mag ×4.

FIG. 2A is a graph that illustrates the level of neural differentiation when retinoic acid and MEDII are and are not present. The first column (ICb−RA) used a standard media (ICb, see Example 2), where a high percentage of non-neural tissue (scored as beating muscle) was observed. The second column (ICb+RA) used a standard media supplemented with all-trans Retinoic acid (RA) at a concentration of 10⁻⁷ M (100 nM) where the ratio of neural tissue to non-neural tissue was increased. The third column used a 50% MEDII conditioned media but without RA supplementation. Again the level of neural tissue produced and the ratio of neural tissue to non-neural tissue was further increased. The fourth column used 50% MEDII supplemented with RA. This produced the highest level of neural differentiation and the lowest level of non-neural tissue as assessed by scoring for cardiomyocyte differentiation.

FIG. 2B is similar to FIG. 2A except the level of neural complexity was scored. A score of 1 to 3 was assigned depending upon the neuronal complexity (number of neurites and networking emanating from the body), with a score of 3 for the most complex. MEDII conditioned media supplemented with RA resulted in more complex neural differentiation than either component separately.

FIG. 3 depicts unstained embryoid bodies at day 12 seeded onto a gelatin matrix at a stage that were scored for neural differentiation and neural complexity as in FIG. 2. The pictures illustrate that RA inhibits non-neural tissue and promotes neural differentiation. A to C are examples of embryoid bodies differentiated on an adhesive surface treated with 50% MEDII. An example of the presence of non-neural tissue is shown in B (arrowhead). D to F. Supplementation with RA decreases the appearance of non neural tissue.

FIG. 4 shows images of adhered embryoid bodies immunostained for the mature neurofilament markers NF200. A to F. Various amounts of MEDII conditioned media was used (10%, 50% and 65%) either supplemented with all-trans Retinoic acid (+RA) or without supplementation (−RA). The pictures illustrate that;
a) RA and MEDII in combination reduces the level of non-neural tissue (beating cardiomyocytes).
b) MEDII alone is not as efficient at reducing non-neural tissue without RA supplementation.
c) RA supplementation stimulates the production of more complex neuronal differentiation in combination with concentrations of 50% MEDII.

FIG. 5 illustrates the derivation and characterisation of a neurosphere population from mouse ES cells grown as embryoid bodies in the presence of 50% MEDII conditioned media with or without RA supplementation that have been dissociated to a single cell suspension and grown in neurosphere media (NSM, see Example 2).
A) FIG. 5 A illustrates a representative field of view of a non-sphere forming population of cells (in this case from a primary passage of EBM¹² +RA). Note the small poorly formed aggregates. 10× magnification.
B) FIG. 5B illustrates a robust sphere formation (in this case from a primary passage EBM¹² no RA) that had formed 4 days after culture in neurosphere media. 10× magnification.
C) FIG. 5C illustrates that spheres that had attached to the bottom of the culture flask (in this case from tertiary passaged EBM¹² no RA supplementation) formed dense networks with neuronal morphology. Note the dense aggregates forming that may indicate sites of new neurosphere formation (arrowhead).
D, E and F) FIGS. 5D, E and F illustrate a single sphere at magnifications 10×, 20× and 40× respectively (in this case from a tertiary passaged EBM¹² no RA supplementation) that had attached and grown for three days in neurosphere media. Similar dense networks formed around the seeded sphere. Note that in F) similar compact clumps of cells are forming that may generate further spheres (arrowhead).
G) FIG. 5G illustrates the immunohistochemistry for NF200 (a mature neurofilament marker specific for differentiated neurones) showing clear labelling of cell bodies and neurites. Magnification 40×.
H) FIG. 5H illustrates the immunohistochemistry for GFAP (Glial/Astrocytic lineage marker) showing a large number of positively stained cells. Magnification is 10×.

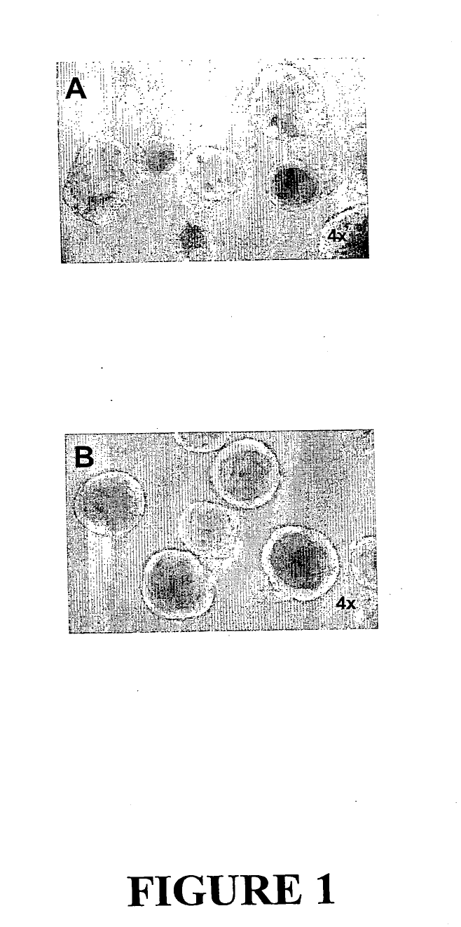

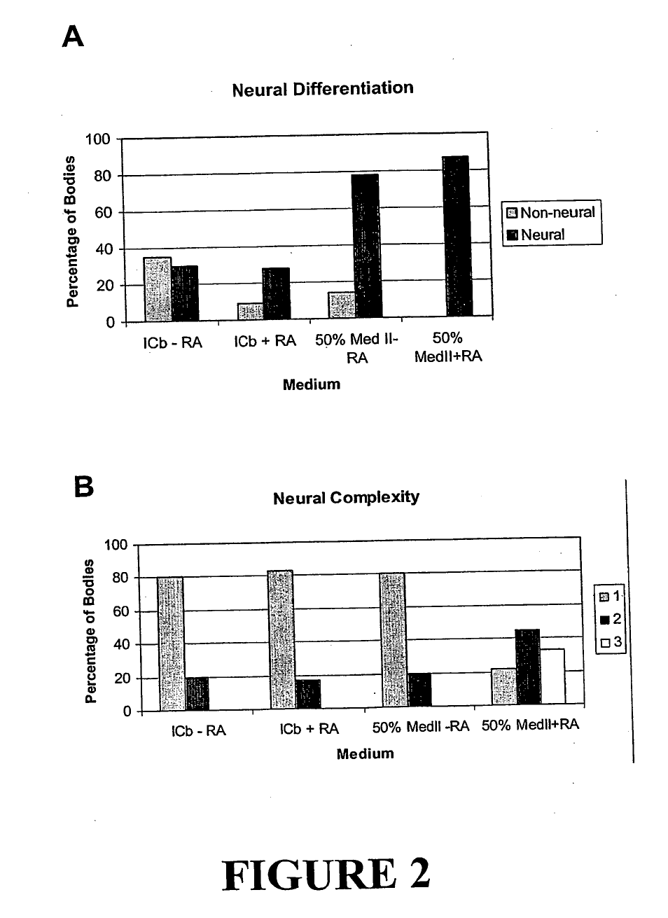

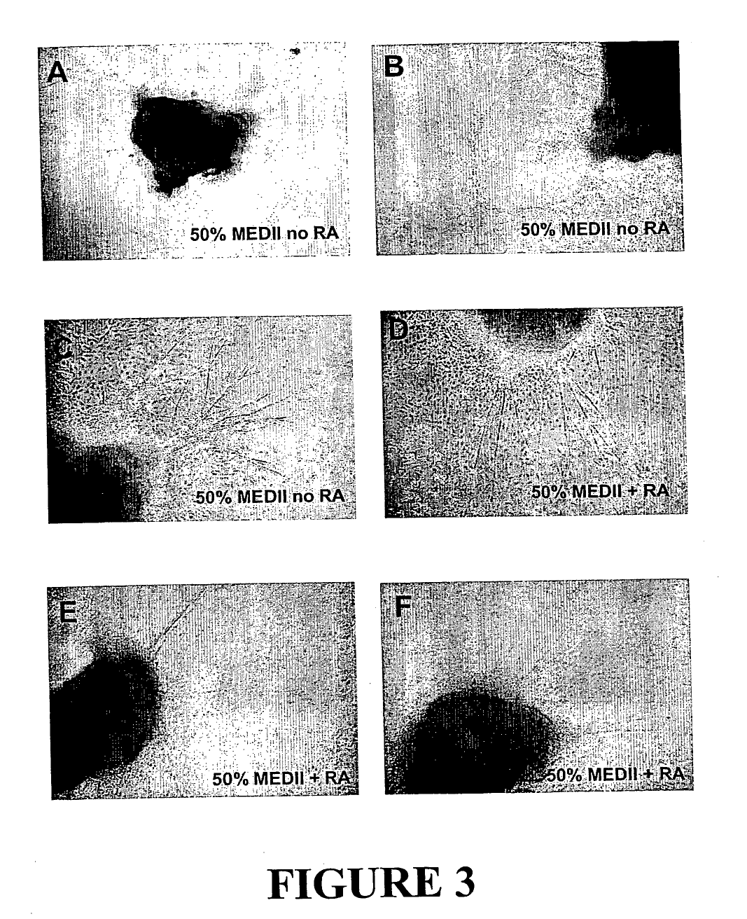

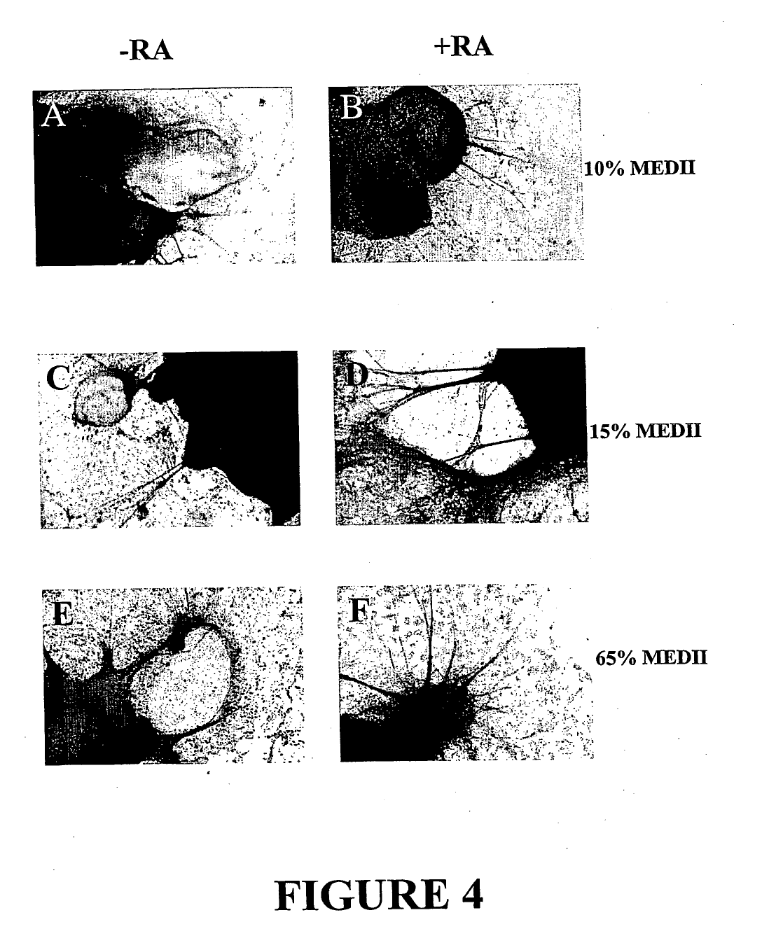

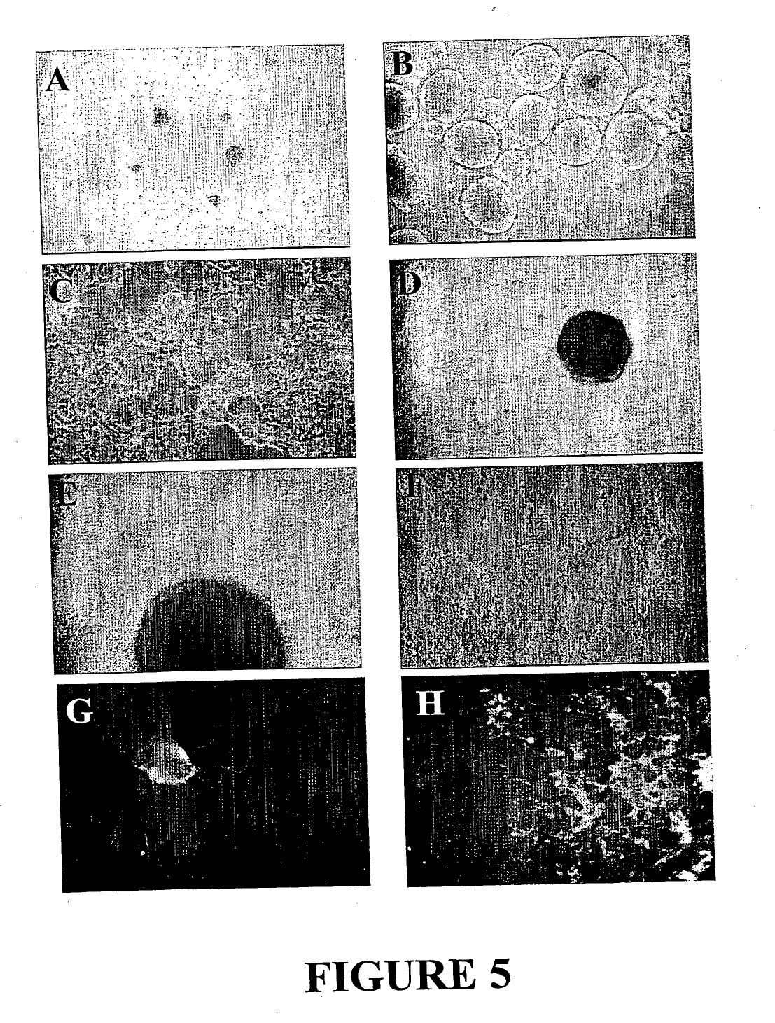

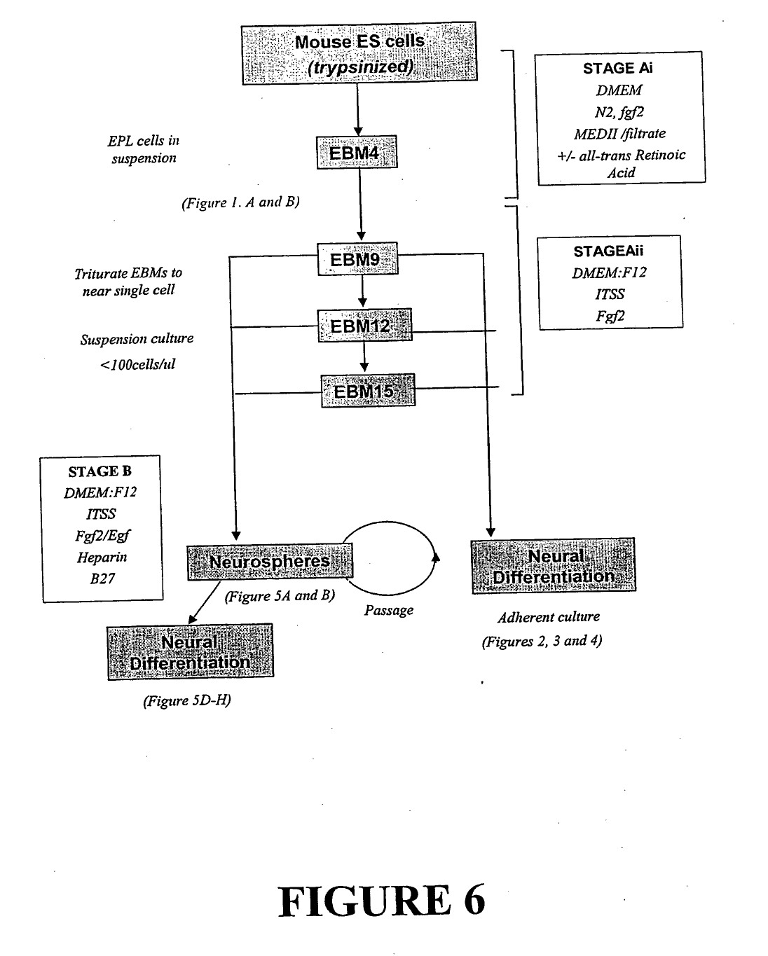

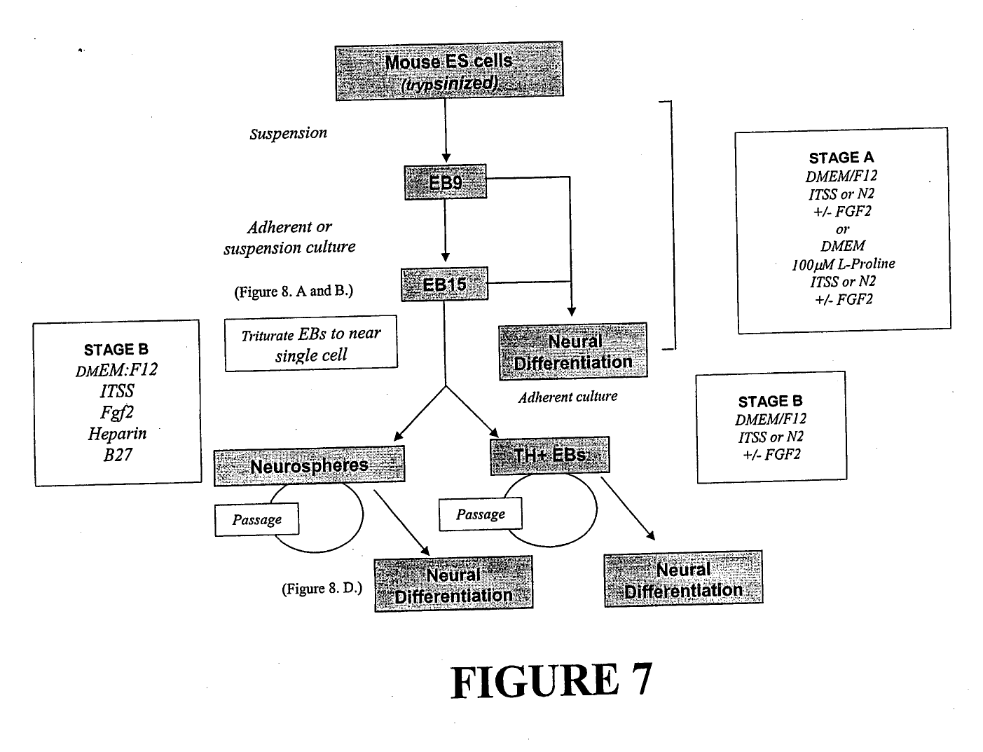

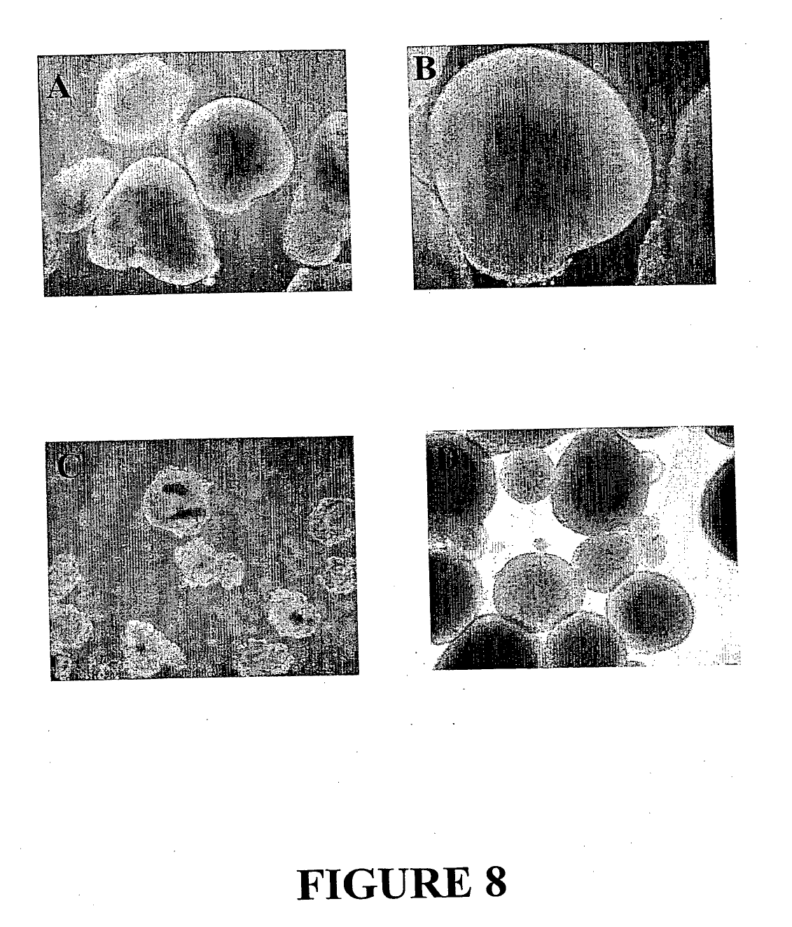

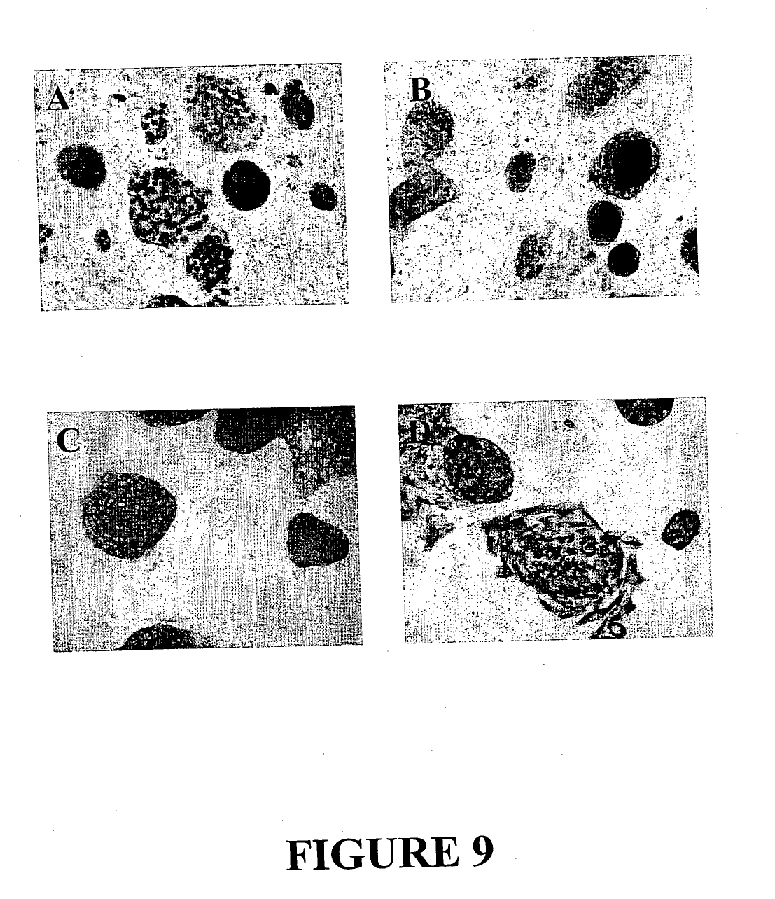

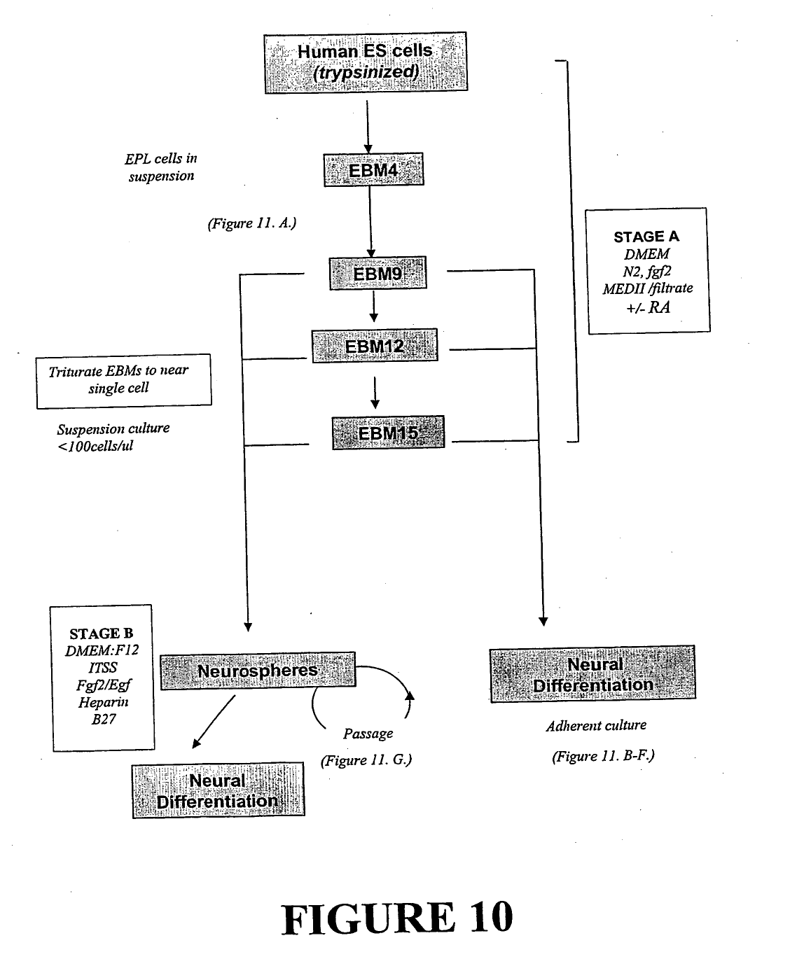

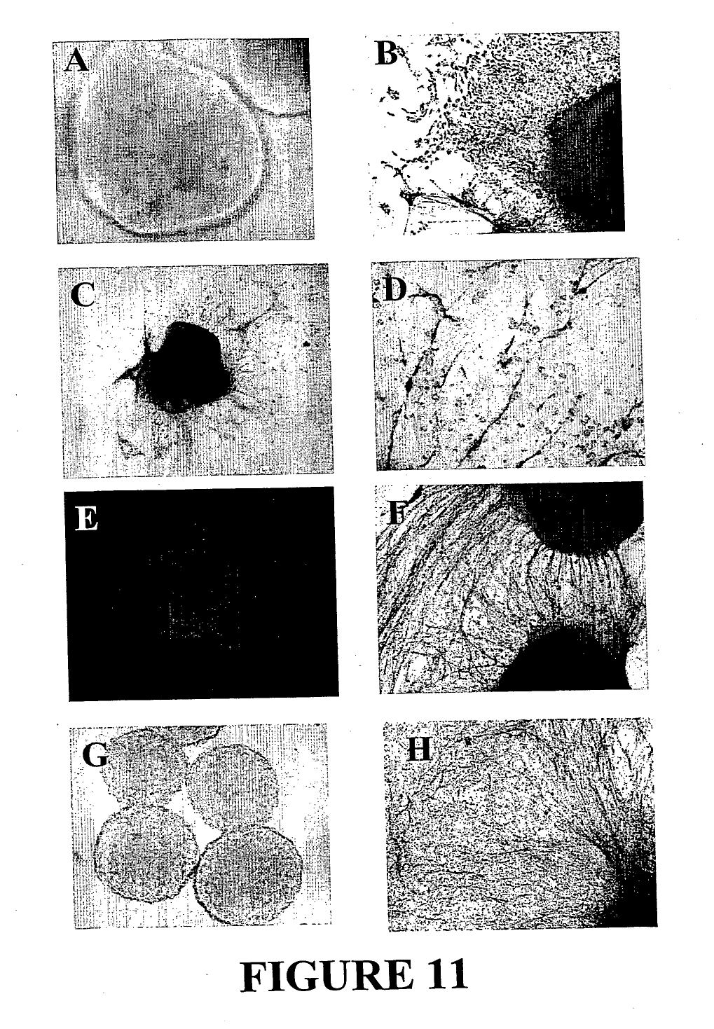

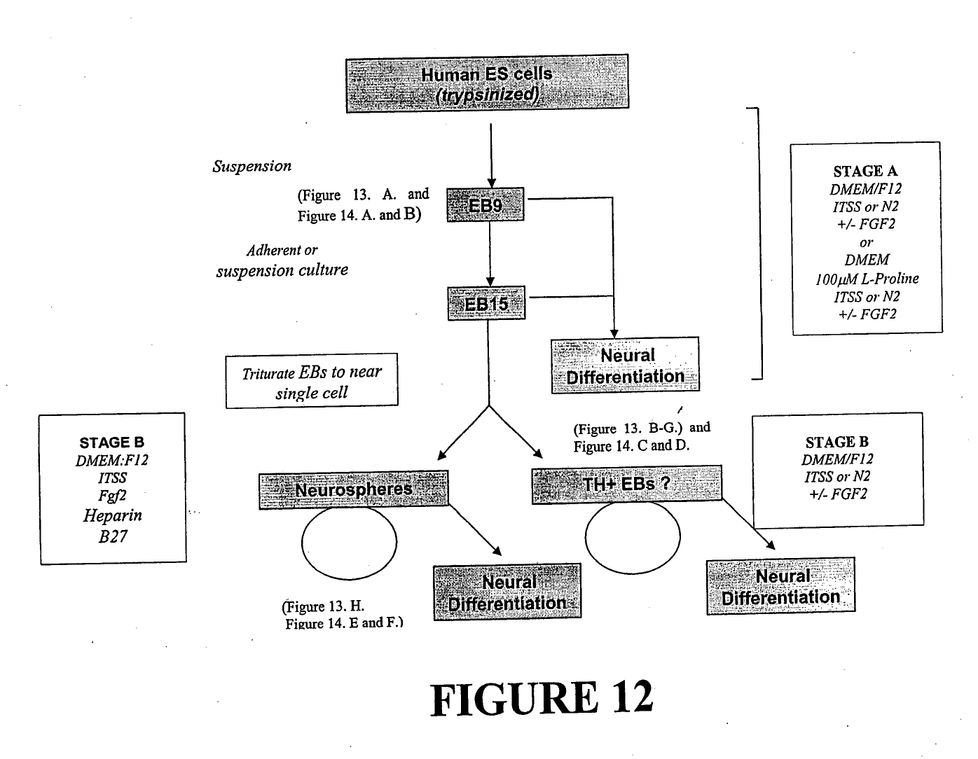

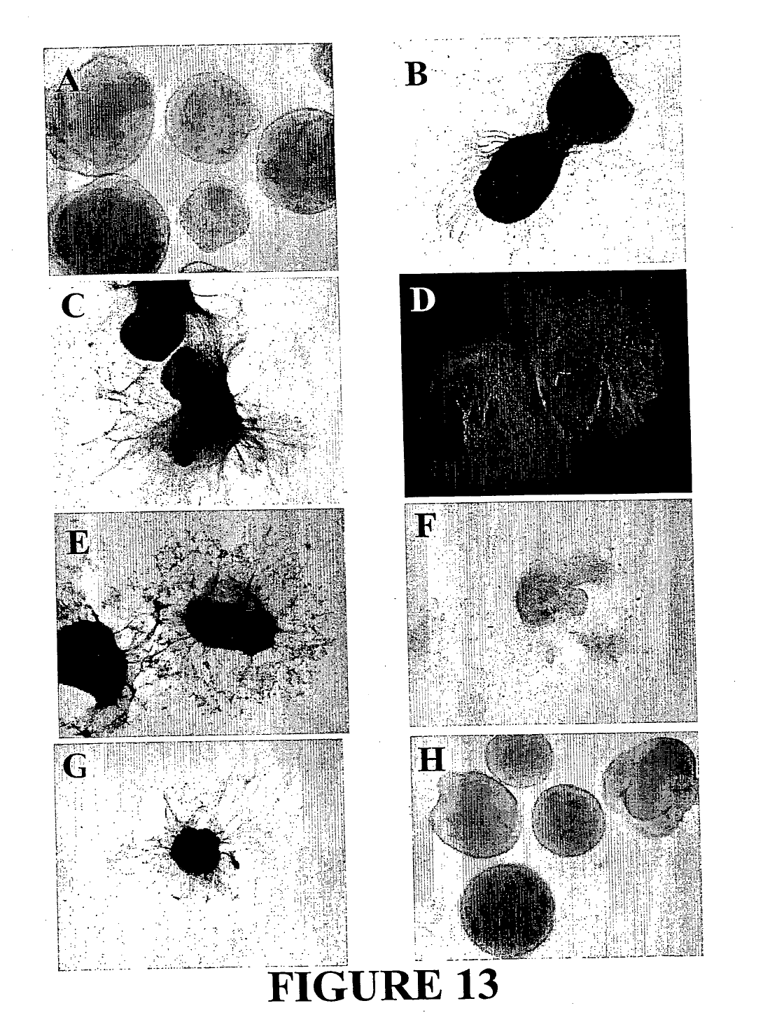

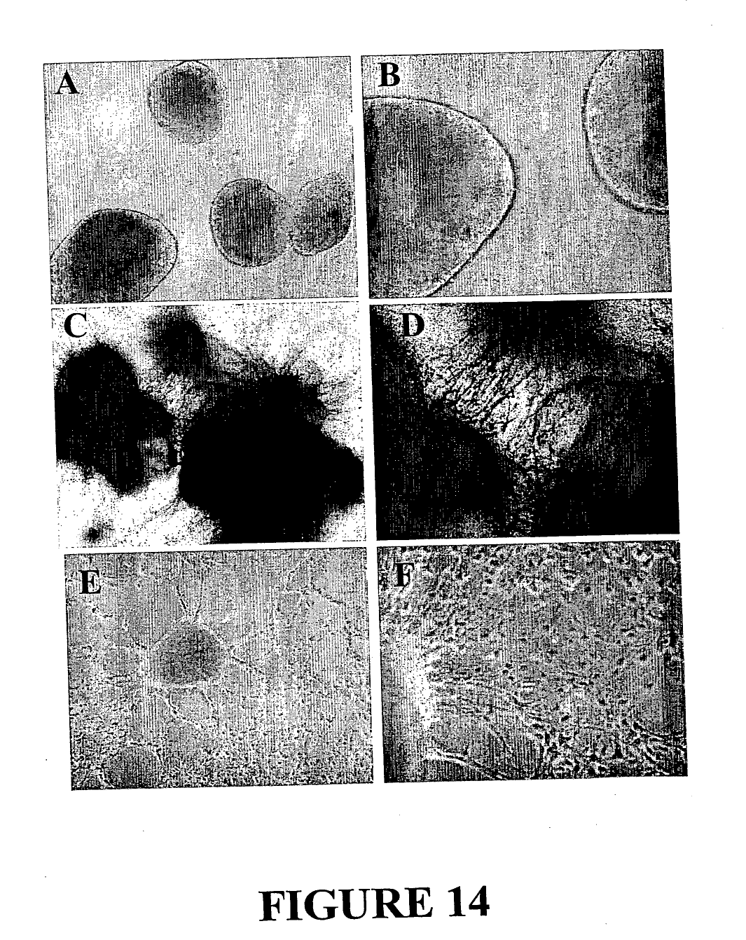

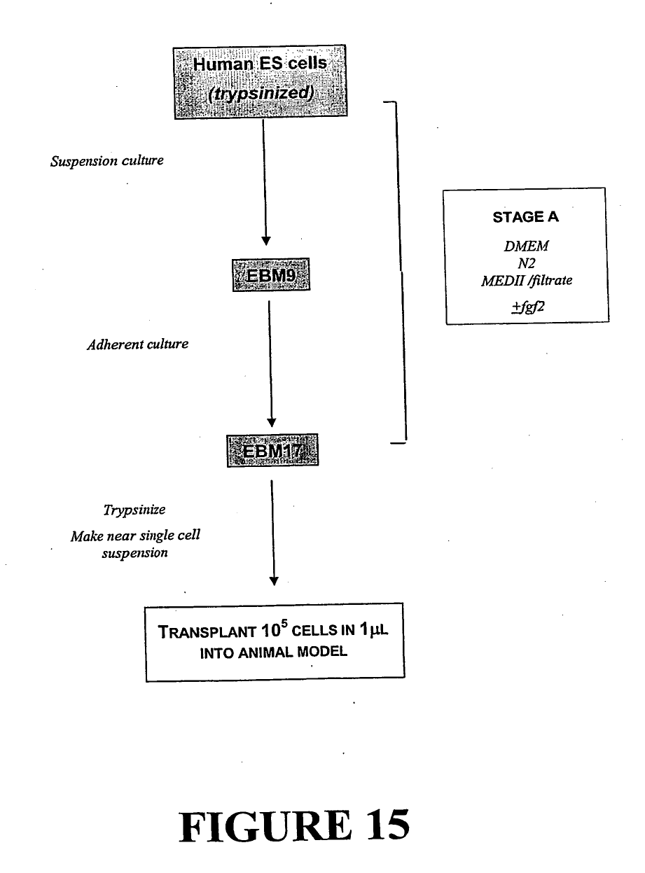

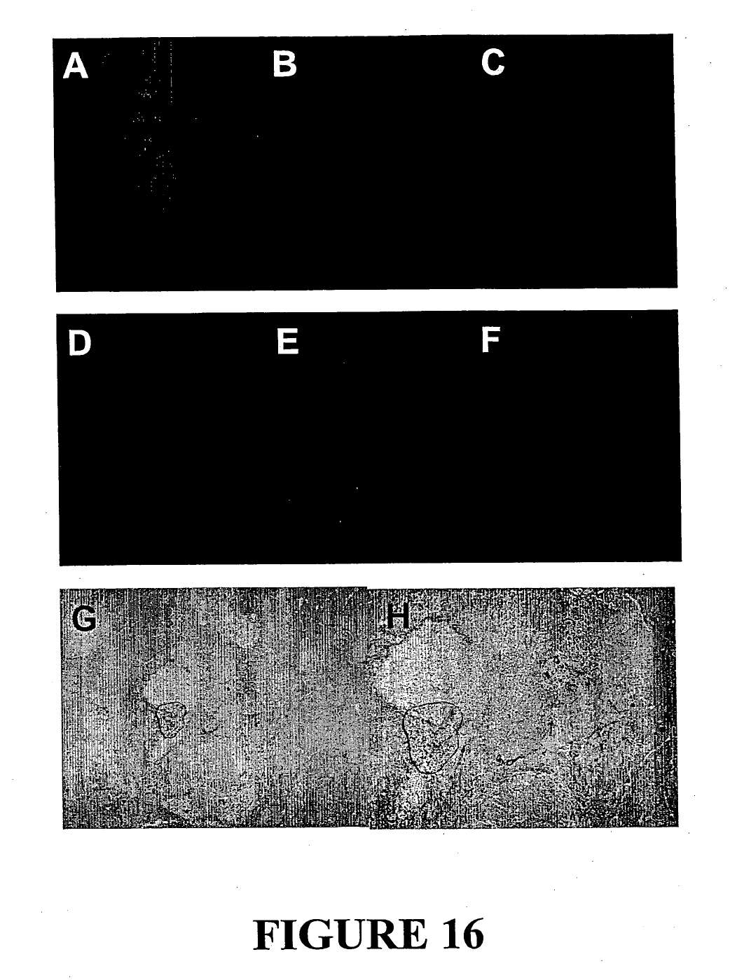

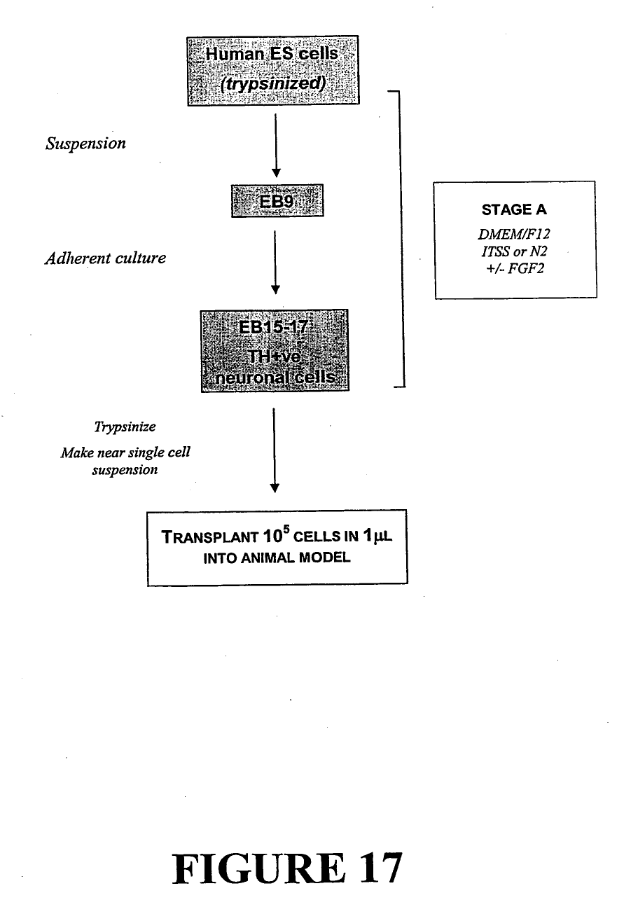

FIG. 6 is a schematic illustrating the production of neurospheres or neuronal cells from mouse ES cells according to the present invention depicted in FIGS. 1 to 5 and Examples 1 and 2. Note that in this process the culture stages have been divided into three phases of growth depending on the media-changes involved. Stages Ai and Aii relate to the initial growth in 50% MEDII for 7 days and a subsequent culturing in a serum free media for a period of up to 9 days. This can be conducted either in suspension or on adherent surfaces. Stage B is the change in media conditions to a neurosphere media when the embryoid bodies are triturated to a single cell suspension prior to seeding at low cell densities in this media. Note that in subsequent examples there are only two stages of media changes (Stage A and Stage B). Figures depicting examples of various stages of this process are shown on the diagram.

FIG. 7 is a schematic illustrating the production of neurospheres or neuronal cells from mouse ES cells in an alternative process to the present invention. The cell aggregate culture medium does not include the conditioned medium MEDII (or its extract) or serum containing ICb media, but does include DMEM Hams F12 media and the supplement N2 or ITSS. Alternatively, included on the schematic is use of DMEM and 100 μM Proline and ITSS or N2 with or without FGF2. Note that a two stage process is followed (Stage A and B). Stage A consists of growth of embryoid bodies in suspension culture optionally followed by a period of adherent culture. Stage B depicts the further growth of embryoid bodies formed during in stage A after their dissociation to a single cell suspension and reseeding in either a neurosphere media or in a media similar to that used in Stage A.

FIG. 8 illustrates the development of mouse ES cells in this media and the subsequent derivation of neurospheres from disaggregated embryoid bodies outlined in FIG. 7. Mouse ES cells (D3) were grown as cell aggregates/embryoid 30 bodies in the basic media DMEM/F12 and ITSS or N2 in low attachment Costar tissue culture plates. A. Cell aggregates/embryoid bodies formed in DMEM/F12 and N2. In this case FGF2 (10 ng/ml) was also present but the morphological aspects of embryoid body formation were the same without FGF. Note the uniform columnar epithelial structure of the body similar to neurectoderm. Mag 10×. Scale bar=200 μM. B A higher magnification of same bodies. Mag 20×. Scale bar=50 μM. C. Mouse ES cells grown in DMEM and N2 and FGF2. Note the loose cellular formation of these cell aggregates and irregular appearance of the cell layer. Robust embryoid body formation with morphological characteristics of neurectoderm did not occur without the presence of F12 media. Mag 10×. Scale bar=200 μM. D. Neurospheres derived from mouse ES cell embryoid bodies grown in the DMEM/F12 and ITSS without FGF2 that were triturated to a single cell suspension and then allowed to form in neurosphere media. These neurospheres have formed after 8 days in suspension culture. Mag 20×. Scale bar 50 μM.

FIG. 9 illustrates the immunohistochemical properties of the SSEA-4 selected Human embryonic stem cells used in the differentiation process outlined in the schematics shown in FIGS. 10 to 17. Human ES cells were initially derived from an SSEA4 selected line and bulk passaged for several passages using collagenase and trypsin (See Example 4 ). Embryonic stem cells depicted are also grown in the absence of LIF and NEAA (Non essential amino acids) and maintained on a mouse embryonic feeder layer. Immunohistochemistry was visualised with HRP-DAB chromogenic reaction. A. Oct4 immunostaining of Human ES colonies. Mag 20×. Scale bar=50 μM B. SSEA4 immunohistochemistry of bulk passaged colonies. Mag 20×. Scale bar=50 μM. C. Alkaline phosphatase expression in Human embryonic stem cell colonies. Mag. 20×. Scale bar=50 μM. D. Nestin immunohistochemistry on Human ES cell colonies using the Nestin rabbit polyclonal (Chemicon). Note that colonies have uniform Nestin expression. Mag. 20×. Scale bar=50 μM.

FIG. 10 is a similar schematic to FIG. 6 illustrating the production of neurospheres or neuronal cells from human ES cells according to the present invention involving the use of serum free MEDII filtrate extract and explained in Example 4. Figures depicting various stages of this process are shown in FIG. 11. Note the in this process, unlike that outlined in FIG. 4, a two stage process is followed with no intermediate change to serum free conditions (FIG. 4, Stage 2ii).

FIG. 11 illustrates the various stages of the differentiation process outlined in the schematic in FIG. 10 using the serum free MEDII filtrate conditioned media. Note that starting population of human ES cells is SSEA4 selected and bulk passaged (FIG. 9) as explained in Example 4. Human ES cells are seeded in suspension into the conditioned media serum free MEDII (Filtrate) to initially form embryoid bodies. A. An example of an embryoid body formed in the serum free MEDII filtrate (8 days in suspension). Note the smooth ectoderm like appearance and the presence of internal neural-tube like structures. Embryoid bodies are also grown in the presence of FGF2. Mag 10×, scale bar=200 μM B. Embryoid are grown until day 9 in suspension, allowed to attach to a laminin coated surface and then grown for a further 8 days in the same media. Cells from the body adhere and spread over the laminin coated surface. In B.cells have been stained for the Nestin antibody, a marker of neural precursors. Many cells show good polarised Nestin+ signal in the cytoplasm. C. Chromogen immunohistochemical staining of an embryoid body 9 days in suspension followed by 8 days adhesive culture with many TH+ cells. Mag. 4×, scale=400 μM. D. Higher magnification of TH+ staining around the body were cells have spread and attached onto the laminin surface. Note the clear distinction of a stained cell (cell body and processes) compared to unstained cells. E. Immunofluorescent detection of TH+ positive cells within the adhered embryoid body. Many TH+ cell bodies are observable inside the body. Mag 4×. Scale=400 μM. F. Adhered EBF bodies also show extensive neuronal outgrowth as determined by chromogenic immunohistochemistry for the mature neurofilament marker NF200. Mag. scale. G. Neurospheres can be derived from these embryoid bodies in suspension from as early as day 9 to day 15. These spheres are passagable at clonal densities (>20 cells/μl). H. Seeded neurosphere plated onto laminin and immunostained for NF200. Mag 10 ×, scale. Passaged spheres were also positive for the Glia/Astrocyte marker GFAP and for TH+ neurones (data not shown).

FIG. 12 is a schematic of an alternative process according to the present invention and similar to that shown in FIG. 7 whereby the cell aggregate culture medium (Stage A) does not include the conditioned medium MEDII filtrate, but does include DMEM and Hams F12 media and N2 or ITSS. Also outlined in the schematic is the use of a media containing DMEM and 100 μM L-proline. This leads to a significant increase in TH positive neural cells in the final neurosphere or embryoid body product.

FIG. 13 illustrates several stages of the differentiation process outlined in FIG. 12 that involves the growth embryoid bodies/cell aggregates grown in DMEM/F12 with either N2 or ITSS. Human ES cells (SSEA4 selected, grown with or without LIF and NEAAs) were allowed to form as cell aggregates/embryoid bodies in the presence of minimal medias (DMEM/F12 or DMEM) supplemented with ITS or N2 and grown with or without FGF2. A. Example of a Human ES cell aggregate/embryoid body grown in DMEM/F12 and ITS. Note the uniform epithelial appearance of the body resembling neurectoderm. Mag 10×. Scale bar=200 μM. B. An embryoid body grown in DMEM/F12, ITSS and no FGF2 in suspension for 9 days and then allowed to adhere on a laminin coated surface for a further 8 days in same media. Immunohistochemistry for TH+ cells revealed that there were substantial numbers of reactive cells extending from the seeded body. Immunofluorescence TH+ signal revealed that their were also large number (˜50%) TH+ cells with the embryoid body (not shown) Scale bar 400 μM. C. An embryoid grown as in B. and immunostained for TH+ but in this case FGF2 was present in the culture media. The presence of FGF2 appears to have had little impact on the generation of TH+ cells. Note the more extensive outgrowth of cells around the seeded body when FGF2 is present in the media. Mag 4×. Scale bar=400 μM. D. Immunofluorescent staining for the immature neuronal marker βIII Tubulin in two embryoid bodies that have adhered close to each other. These embryoid bodies were grow for 9 days in suspension followed by 8 days of adhesion on laminin in a media consisting of DMEM/F12, ITSS and no FGF Mag 10×. E. Chromogenic immunostaining for the mature neurofilament marker NF200. Embryoid bodies were grown for 9 days in suspension and 8 days adherent culture in a media consisting of DMEM/F12, ITSS and no FGF2. Mag ×4. Scale bar=400 μM. F. An example of an embryoid body grown in the media DMEM, N2 and no FGF for 9 days in suspension followed by 8 days adhesion on laminin and immunostained for TH+ cells. Very few TH+ cells are visible emphasising the requirement for F12 (or a component of the F12) for the differentiation of TH+ cells Mag ×4. Scale bar=400 μM G. An embryoid body grown as in F. and immunostained for the mature neurofilament protein NF200. Many NF200 positive cells are present suggesting that the absence of TH+ neurones is not due to an inability of neuronal cells to differentiate under these growth conditions. Mag ×4. Scale bar=400 μM. H. Neurospheres were able to be derived from embryoid bodies grown in DMEM/F12 and N2 or ITSS without FGF2. This picture shows neurospheres that have formed in neurosphere media after 16 days growth. The embryoid bodies/cell aggregates from which these spheres were derived were grown in DMEM/F12, ITSS and no FGF2. Passaged neurospheres were also positive for the neurofilament marker NF200. Mag ×10. Scale bar=200 μM.

FIG. 14 illustrates several stages of the differentiation process outlined in FIG. 12 that involves the growth of embryoid bodies/cell aggregates in the DMEM and 100 μM L-Proline and N2 or ITSS. Proline is component of Hams F12 media (300 μM). In DMEM/F12 the concentration of Proline is 175 μM. The following pictures in these examples show the effect of growing Human embryoid bodies in the following conditions; DMEM, 100 μM L-Proline and either ITS or N2 supplements with or without FGF2. A. Embryoid bodies formed after 9 days in suspension Mag. 10×. Scale bar=200 μM. B. Higher magnification (20×) of same cell aggregates/embryoid bodies. Mag 20×. Scale bar=50 μM. C. Embryoid bodies grown in suspension for 9 days and then seeded onto poly-L-ornithine/laminin for a further 8 days and allowed to adhere and grow. Immunostained for TH+ cells. Note that under these conditions TH+ cells can form at relatively high numbers. Compare this with embryoid bodies grown in DMEM and ITS or N2 only (i.e no F12 or Proline supplementation) form very few if any TH+ cells. D. Higher magnification depicting TH+ fibres emerging from the Proline treated embryoid body. E. Neurospheres can also be derived from these DMEM/Proline embryoid bodies. Example shows a sphere that has been allowed to attach to a laminin coated surface and is starting to differentiate. Mag 20×. F. Higher magnification (20×) of attached sphere. Note the dense network of cells that have arisen form the seeded sphere with neuronal morphology (extensive neurites and intercdnnectivity). Passaged neurospheres were also positive for the neurofilament marker NF200.

FIG. 15 is a schematic illustrating an alternative method according to the present invention, in which human ES cells are grown in the presence of the conditioned medium MEDII filtrate to produce a cell population including a proportion of TH+ cells. Note that in this process only a Stage A culturing was followed with embryoid bodies formed in suspension culture for 9 days followed by an 8 day period on a laminin coated adhesive surface.

These cells are then transplanted into an animal model.

FIG. 16 illustrates the in vivo differentiative behaviour cells that have been produced as outlined in the FIG. 15 schematic after an 8 week incubation period in the adult Rat Striatum. Illustrative examples of the differentiation that occurs following the implantation of cells into the 6-OHDA lesioned adult Rat Striatum described in the FIG. 15 schematic. Human embryonic stem cells underwent a differentiation procedure that involved differentiation in a MEDII filtrate conditioned media. This involved 9 days in suspension followed by 8 days adherent culture on a Laminin coated surface. A. B. and C. An example of a Rat (N 274) that had received an implant of cells as outlined in FIG. 15. Implanted human cells express the neuronal marker GFAP. A, GFAP and astrocyte/glial lineage marker, B, DAPI a non-specific nuclear marker and C an Alu DNA probe in situ specific for detection of human cells are shown Mag ×4. This example shows that implanted human cells are able to differentiate to glia. D, E and F. An example of a Rat (N278) that received an implant of cells as outlined FIG. 15. Implanted human cells express the neural precursor marker Nestin. D, Human specific Alu DNA probe in situ, B, Nestin immunohistochemistry and C, a general nuclear marker DAPI. Mag 10×. G and H. Immunohistochemistry for the detection of TH+ cells using chromogens. A Rat (N278) with implanted cell that express the dopaminergic neurone lineage marker Tyrosine Hydroxylase. A small cluster of TH+ cells can be seen in G (10× Mag) and H (20× Mag, arrowhead) with clearly staining cell bodies.

FIG. 17 is a schematic illustrating a still further embodiment of the method according to the present invention in which human ES cells are grown in a culture medium containing Hams F12 to produce a cell population including a high proportion of TH positive neuronal cells.

These cells are then transplanted into an animal model.

### EXAMPLE 1

Single cell suspensions of mouse ES cells were cultured for 4 days in standard culture media (ICb) or in the presence of 50% MEDII. The standard conditioning media (ICb) contains 90 ml DMEM, 10 ml of foetal calf serum, 1 ml glutamine (0.1M stock at 1/100 dilution) and 100 ul of β-Mercaptoethanol (0.1M stock at 1/1000 dilution). The culture process involved media changes on days 2 and 4 involving a ½ splitting of the cell aggregates. On the fourth day the cells received a 100 nM (10⁻⁷ M) of all-trans Retinoic acid (RA) and 50% MEDII conditioned media. This media change occurred every day for three days (i.e to EBM⁷). By the end of this period the morphological differences in treatment when supplemented with combinations of 50% MEDII and/or RA were apparent. On day 8 the bodies were transferred to serum free media conditions (see example 2) with FGF2 (10 ng/ml) and a final dose of RA (this completed the 4−/4+ RA treatment). On day 9 the cells were cultured in serum free media with FGF only.

This example was used to produce the bodies referred to in the above figures with RA and MEDII either being present in the amounts identified or absent as referred to in the figure legends (FIGS. 1-6).

Two other doses of RA were tried, 1 μM and 10 nM, but the effect was not as uniform as seen with 100 nM RA.

### EXAMPLE 2

Preparation of EBs and EBMs from Mouse ES Cells

Mouse D3 ES Cells were maintained on gelatin free tissue culture plates and passaged every three to four days. ES Culture media was 10% s FCS, DMEM, 1 μM β-Mercaptoethanol, 1 mM Glutamine, 1000 U/ml mouse LIF (ESGRO). Embryoid bodies were formed from ES cells by rinsing ES cell colonies with PBS twice, treated with Trypsin/EDTA for 1 minute, triturated and blocked with an equal volume of FCS before being centrifuged and resuspended and counted.

Embyroid bodies (EBs) were formed by seeding the single ES cell suspension at 1×10⁵ cells/ml in ICb media (see example 1). Bodies were allowed to aggregate for two days and then split 1:2 in ICb media (EB²), cultured for a further 2 days and split again 1:2 (EB⁴) and then cultured for three further days with daily changes of media (EB⁷). Culture conditions were then changed to Serum-Free media (50% DMEM, 50% Hams F12 (Gibco, BRL), 1×ITSS (Boehringer Mannheim) and 10 ng/ml FGF-2 (Peprotech Inc.) for a further period up to 8 days (EB¹⁵).

For MEDII conditioned embryoid bodies (EBMs), a single cell suspension of ES cells was seeded at 1×10⁵ cells/ml in ICb supplemented with 50% MEDII. Bodies were allowed to aggregate for two days and then split 1:2 in 50% MEDII media (EBM²), cultured for a further 2 days and split 1:2 (EBM⁴, EPL cells in suspension) and then cultured for three further days with daily changes of 50% MEDII media (EBM⁷). Media was then changed at this stage for further culturing to Serum-Free media as for standard embryoid bodies for a further period up to 8 days (EBM¹⁵)

All-Trans Retinoic Acid Supplementation

For treatments involving all-trans Retinoic acid (SIGMA) a 4−/4+culture supplementation was followed. Briefly, both EB and EBM cell aggregates were allowed to form in appropriate media for four days and were then supplemented with 100 nM all-trans RA with a daily media change thereafter for a further 4 days.

Generation of Neurospheres

Both EBs and EBMs were triturated to a near single cell suspension after culturing for periods of 7, 9, 12 and 15 days (7 days in 50% MEDII followed by an appropriate number of days in Serum Free media). Two methods of trituration, either mechanically or using trypsin yielded similar results. Cells were seeded at approximately 10 to 20 cells/μl of media into 10 mls of neurosphere media (DMEM:F12, 10 ng/ml FGF₂, 10 μg/ml heparin (SIGMA), 1/50 B27(GIBCO), 1/100 pen/strep, 1/100 ITSS) in a T75 culture flask. Cultures were maintained in this media with a 50:50 change of fresh media after 7 days and cultured for 10 to 12 days. Sphere formation was readily apparent after three days in culture and robust spheres had formed by day 7. One population of spheres derived from EBM¹² (no RA) aggregates was passaged and grown for a further two passages to yield tertiary spheres. These spheres were seeded onto Poly-L-ornithine/fibronectin coated chambers slides (Nunc) and cultured in neurosphere media for 3 days before fixing and processing for three representative neuronal lineage immunohistochemical markers (NF200, GFAP and O4).

A cell suspension was also prepared from tertiary passaged EBM¹² (No RA) spheres and 1000 cells stereotactically injected (1000 cells/μl) into the striatum of six Sprague-Dawley rats. Two rats were harvested 2 weeks post engraftment and 4 rats 4 weeks post engraftment. Rats were maintained under conditions of immunosuppression, using cyclosporin A. Grafted mouse cells were detected using a mouse DNA satellite marker (data not shown).

Results

Formation of neurospheres differed dramatically between single cell suspensions generated from either ES cell aggregates (embryoid bodies) grown in either ICb (EBs) or in 50% MEDII (EBMs). From Table 1, ES cell aggregates formed in ICb followed by periods of Serum starvation exhibit poor sphere forming capacity even when treated with RA. In contrast, a MEDII dependent effect was observed in cell aggregates that had formed in 50% MEDII (EBMs) followed by a period of serum starvation. Robust sphere forming capacity was clearly seen in EBM¹² aggregates with clear sphere formation visible after 3 to 4 days in neurosphere culture media. The capacity for sphere formation seemed to be diminished in EBMs either side of this time frame. An effect of RA was observed such that sphere-forming capacity seemed to emerge earlier at EBM⁹ and showed a decrease with later stage EBMs. In both cases, robust sphere forming capacity was only seen in cells derived from bodies that had been conditioned in 50% MEDII.

TABLE 1

| | Day 7 | Day 9 | Day 12 | Day 15 |
|---|---|---|---|---|
| | (50% | (50% | (50% | (50% |
| | MEDII or | MEDII or | MEDII or | MEDII or |
| | IC:DMEM | IC:DMEM + | IC:DMEM + | ICDMEM + |
| | only) | 2 days SF) | 5 days SF) | 8 days SF) |
| EB | − | − | − | − |
| EB + RA | − | − | − | − |
| EBM | + | + | +++ | ++ |
| EBM + RA | + | +++ | + | + |

EB = embryoid body,
EBM = embryoid body cultured in MEDII,
RA + 100 nM all trans retinoic acid.
(− no or very poor sphere formation,
+, ++ and +++ indicate robustness of sphere formation,
+ poor sphere forming capacity,
++ moderate sphere forming capacity,
+++ high sphere forming capacity)

Sphere formation was observed after two further passages of EBM¹² (No RA) spheres that were mechanically passaged and reseeded at a 10 to 20 cells/μl density in neurosphere media. During the passaging of these cells it was noted that dense networks of cells formed on the bottom of the flask where spheres had attached. These structures exhibited extensive neural morphology and extensive networks of neurites were observed. Dense clusters of cells appeared and were likely to act as a source of more spheres. Spheres from these tertiary passaged spheres were seeded onto glass chambers slides and allowed to grow for three days before processing for immunohistochemistry. These single spheres grew to form similar extensive networks of cells with dense regions that seemed to be forming more spheres. Marker analysis revealed that there were large numbers of GFAP+ cells (an astrocytic lineage marker) whereas NF200 positive neurons formed at moderate levels while O4 positive oligodendrocytes were present at low levels (data not shown). The seeded spheres were therefore capable of producing cells of all three neuronal lineages after three passages at clonal cell densities and therefore provide evidence of self-renewal and multipotency.

The results are presented in FIGS. 1 to 5.

This process is outlined in the schematic presented in FIG. 6.

A preliminary in vivo analysis of low cell number grafts into the rat Striatum revealed detectable mouse cells that line the needle tract of the injection site. No obvious signs of gross teratoma formation were visible and the number of detectable cells was low (10 to 20 per 10 μM section through graft site, data not shown).

### EXAMPLE 3

Example 2 was repeated but following the schematic set out in FIG. 7. In contrast to Example 2 a two-stage process was followed with cells grown as aggregates/embryoid bodies in the one media (Stage A) prior to disaggregation for Stage B growth conditions. Mouse ES cells were separated by treatment with trypsin and the single ES cell suspension seeded in a cellular aggregate culture media (DMEM:F12 and either N2 or ITSS) that was free of serum. The addition of 10 ng/ml FGF2 and/or 100 nM dose of RA is optional. Alternatively the cell aggregates/embryoid bodies can be formed in the presence of DMEM and 100 μM proline with either N2 or ITSS and optionally with FGF2 and RA. Cell aggregates/embryoid bodies were allowed to form in Costar low attachment tissue culture dishes for a period of up to 15 days in suspension culture (Stage A) and then were triturated to a single cell suspension using trypsin dissociation.

Cells were then seeded at a concentration of less than 100 cells/μL in neurosphere media (DMEM:F12, 10 ng/ml FGF2, 10 μg/ml heparin (SIGMA), 1/50 B27(GIBCO), 1/100 pen/strep, 1/100 ITSS) in a T75 culture flask. Cultures were maintained in this media for 14 to 21 days (Stage B) and the neurospheres so formed can be maintained by passaging in the same media.

The neurospheres formed can be seeded onto poly-L-ornithine/laminin coated plates and allowed to adhere and differentiate. Optionally in this culture stage, neurospheres can be maintained in media containing combinations of RA, 50% MEDII and L-proline. In an alternative treatment, during stage A, the embryoid bodies (EB⁹) can be seeded onto poly-L-ornithine/laminin coated culture plates and cultured for 6 to 8 days to permit neuronal differentiation.

In an alternative treatment, embryoid bodies cultured from stage A can be triturated and resuspended in a minimal media (DMEM/F12 and N2 or ITSS). Optionally this media can also include combinations of FGF, MEDII, RA and L-proline. The aggregates formed can also be seeded onto poly-L-ornithine/laminin coated plates and allowed to adhere and differentiate.

Examples of cell aggregates/embryoid bodies and neurospheres that can be formed are presented in FIG. 8. A to D.

### EXAMPLE 4

In this example the method illustrated in Example 2 was essentially repeated utilising human ES cells, with the following differences. For human ES cells the MEDII conditioning was conducted using the Filtrate (<10 Kda fraction) of serum-free MEDII. Secondly human cell aggregates were formed as suspension bodies in 50% serum-free MEDII Filtrate for a period of up to 15 days with no change in media at EBM⁹. Neurospheres were then formed from embryoid bodies after disaggregation to near single cells.

Culture and Passage of Human ES Cells

SSEA4 selection of Human ES cells was carried out (BresaGen Inc. Athens, Ga., USA) using magnetic bead separation and these initially sorted cells have been used in the bulk passaging protocol for these experiments. ES cells cultured onto Mitomycin C treated mouse Embryonic Fibroblast feeder layer (MEFs). A seeding density for MEFs of 1.2×10⁶/35 mm TC dish was used and MEFs were not used until they were 3 days in culture. Two ES cell culture media were used for maintaining Human ES cells; Complete (+LIF +NEAA) and Incomplete (−LIF −NEAA) via bulk passaging (Trypsin dissociation). Immunohistochemical characterisation of Human ES cell colonies grown in these two media indicate that in terms of the expression of pluripotent markers (Alkaline Phosphatase, Oct4 and SSEA4) LIF and NEAA are not necessary for Human ES cell culture. Human ES cells passaged in this manner are also Nestin positive (See FIG. 9).

HES Cell Culture Media

HES culture medium was prepared as shown below.

| | | |
|---|---|---|
| DMEM/F12 (1X, GibcoBRL#11320-033) | 80 | ml |
| KSR (GibcoBRL#10828-028) | 20 | ml |
| β-Mercaptoethanol (0.1M) | 0.1 | ml |
| Glutamine (100X, GibcoBRL#25030-081) | 1 | ml |
| Penicillin/Streptomycin (100X, GibcoBRL#15070-063) | 1 | ml |
| bFGF/FGF-2 (SIGMA 25 ug/ul) | 4 | ng/ml |
| | | final |
| | | conc |

Passaging of HES Cells

Passaging of Human ES cells was conducted every 3 to 4 days. Seeding density used was 3×10⁵cells/3 cm TC dish on MEFs. Collagenase treatment was used to firstly remove the MEFs feeder layer followed by a gentle trypsin treatment for single cell disaggregation of ES cell colonies. To prepare a 5 ml Collagenase solution 5 mg of Collagenase Type IV (GibcoBRL#17104-019) was completely dissolved in 5 ml DMEM/F12 medium (GibcoBRL#11320-033) to give a working stock of 1 mg/ml (between 150-250 units of enzyme/ml). Filter sterilisation was conducted using a 0.20 um filter (Sartorius#0297) and a 10 ml syringe (Becton Dickinson#302146). This solution is stable at 4° C. for 1 week. 0.05% Trypsin/0.53 mM EDTA in HBBS (1×, GibcoBRL#25300-054)

SF MEDII/Filtrate Preparation

MEDII conditioned medium was prepared as described in WO 99/53021. The filtrate fraction of MEDII was prepared by ultrafiltration through a 10⁴ Mᵣ cut-off membrane (Centricon-3 unit; Amicon) as described in WO 99/53021. Essentially the filtrate contained molecules less than 10⁴ Mᵣ.

Formation of Human Embryoid Bodies

Collagenase/trypsin passaged ES cells were prepared as a single cell suspension and seeded at a density of 150 cells/μl in low attachment TC dishes (Costar). Cell aggregates were split 1:3 at day 2 and possibly at day 3 if required. Cultures were feed daily for 9 days and on day 9 bodies were transferred to poly-L-ornithine/laminin coated 24 well trays in 0.5 ml of medium if adhesive culture was to be conducted. Another 0.5 ml media was added to each well after 24 hours incubation. Adhered cultures or suspension cultures were fed daily for a further 8 days.

Adhesive Culture for Neural Differentiation

Embryoid bodies or neurospheres/aggregates are allowed to settle onto a coated surface to allow differentiation to occur (4 to 8 days). The coating can be on a plastic surface in either a tray or a coated coverslip.

Poly-L-Ornithine/Laminin Coating

300 μl of poly-L-ornithine 0.01% solution (Sigma Cat # P4957) was added directly from bottle into each well of a 24 well tray or a 4 well tray. Trays were sealed with parafilm and incubated overnight at 4° C. Wells were rinsed 3× with sterile MQ water. Laminin (Sigma Cat# L20-20) was diluted from a 1 mg/ml frozen stock to 1 μg/ml in sterile MQ water. 300 μl of laminin (1 μg/ml) was added to each well. Trays were sealed with parafilm and incubated overnight at 4° C. Wells were rinsed 3× with sterile MQ water and then once with 1× PBS. Trays were stored with PBS at 4° C. for up to 2 to 3 weeks. Prior to seeding wells were rinsed with 1× medium by adding 1 ml of seeding media and incubating at 37° C., 5% CO₂ to equilibrate.

Preparation of Human Neurospheres

Trypsin-EGTA Disaggregation of Embryoid Bodies. A 10 ml pipette was used to transfer bodies to a yellow capped tube. Media was aspirated and 5 ml Sigma PBS added. Bodies were allowed to settle and the PBS was aspirated and 1.25 ml of EGTA (pH 7.5) was added to the tube and bodies were soaked for 5 mins at room temperature. Solution was aspirated and 0.5 ml trypsin was added to bodies for 30 secs. Disaggregation of the bodies was carried out by gently pipetting them up and down with a P1000 Gilson pipette until there are no large cell clumps. 0.5 ml FCS was then added and the disaggregation continued until solution was uniformly dispersed. 10 ml DMEM+5% FCS was then added and cells were spun at 300 rpm for 1 min to remove clumps. The supernatant was transferred into a fresh yellow capped tube 15 ml conical bottom tube and cells pelleted at 1200 rpm for 4 mins. The cell pellet was then resuspended in 100 μl of DMEM +5% FCS and a count of viable cells was performed. Dissociated cells were then seeded into a T25 flask @ 50-100 cells/μl in 6 mls of neurosphere media (NSM; DMEM/F12, B27 1:50, ITSS 1:100, Heparin (10 mg/ml) 1:1000, FGF2 ((25 mg/ml) 1:5000 dilution) and spheres allowed to form over a two-three week period. NSM was changed 50:50 every 4 days.

Passaging of Neurospheres

Disaggregation of neurospheres was conducted either using the trypsin dissociation method described above for the preparation of neurospheres or using a mechanical trituration method as follows. Using a 10 ml pipette, spheres were transferred to a 15 ml yellow capped conical bottom tube. Spheres that had attached to the flask were gently dislodged with 5 mls fresh media and added to the tube. Spheres were pelleted by centrifugation. Supernatant was removed, leaving behind approximately 200 uL and the pellet gently triturated approximately 150× using a p200 pipetman. 5 ml of culture medium was added and centrifuged gently to remove debris. Supernatant was removed and cells were gently dissociated 10-20× to disaggregate the pellet. A viable cell count was done and cells were reseeded at 1×10³ cells/cm² (equivalent to ˜4 cells/μl).

Results

1. MEDII Filtrate

In the presence of MEDII filtrate, neurospheres were derived from EBM⁹s. If filtrate was omitted, derivation of neurospheres from EBMs was delayed until EBM^{12-15.}

2. Neurospheres

Neurospheres contained neuronal cells (NF200+ve). Neurospheres also included glial cells (GFAP+ve). TH+ neurones were also present after passaging.

### EXAMPLE 5

Essentially the process using mouse ES cells, outlined in Example 3 was repeated with some modifications using human ES cells. A two stage protocol was followed as outlined in FIG. 12.

Human ES cell culture, cell aggregate/embryoid body formation and adherent culture was essentially as described in Example 4. Cultured human ES cells expressed the same characteristics as described in FIG. 9.

Basic Media (DMEM/F12 and ITSS or N2).

Embryoid bodies/neurospheres from human ES cells were grown without the use of MEDII conditioned media. Media and supplements used were Hams DMEM/F12 (Gibco Cat # 11320-033), ITSS (Gibco Cat#17502-048) and N2(Gibco Cat#41400-045). Media did not contain HEPES.

Comparison of Basic Media with Supplements Vs.

The ability of basic media with supplements (DMEM +N2 or ITSS) to promote neural differentiation of hES cells was compared with medium that included F12: (DMEM:F12 (1:1) +N2 or ITSS)

Initial results showed that embryoid bodies can form in either of these basic media even without FGF2. Immunohistochemistry for NF200 reveals that under both media conditions with either supplement neurones can form (FIG. 13. E and G). Furthermore, without the addition of a mitogen such as FGF2 there are still proliferating cells (Ki67 positive cells, not shown). An important distinction between the two media is that TH+ positive cells are present in large numbers (˜50%) in DMEM/F12 and either supplement (N2 or ITSS) but not in DMEM only with either supplement (FIG. 13. Compare B and C with F).

Trypsinised Human ES cells were seeded at approximately 10 to 20 cells/μl of media into 10 ml of neurosphere media (DMEM:F12, 10 μg/ml heparin (SIGMA), 1/50 B27(GIBCO), 1/100 pen/strep, 1/100 ITSS) in a T75 culture flask. 10 ng/ml FGF2 was optionally added but the culture medium was preferably mitogen-free (no FGF2). Cultures were maintained in the media for 9 days after which the embryoid bodies were optionally transferred to poly-L-ornithine/laminin plates and cultured in the same media for a further 6 days. The embryoid bodies so formed (EB¹⁵) whether from adherent or suspension culture, were then triturated to near single cell form and used for either transplantation or for the formation of neurospheres/cell reaggregates.

Formation of neurospheres was achieved as described in Example 4. The neurospheres formed were seeded onto poly-L-ornithine/laminin-coated plates and allowed to adhere and differentiate. Optionally in this culture stage, neurospheres can be maintained in media containing combinations of RA, 50% MEDII or filtrate, and L-proline. In an alternative treatment, during stage A, the embryoid bodies (EB⁹) can be seeded onto poly-L-ornithine/laminin-coated culture plates and cultured for 6 to 8 days to permit neuronal differentiation.

In an alternative treatment (Stage B), neurosphere formation was achieved when embryoid bodies formed from stage A were triturated and resuspended in a minimal media (DMEM/F12 and N2 or ITSS). Optionally this media can also include combinations of FGF, MEDII, RA and L-proline. The aggregates formed can also be seeded onto poly-L-ornithine/laminin coated plates and allowed to adhere and differentiate.

Results of these experiments are shown in FIG. 13.

Results

1. F12 Media

In the presence of F12 media embryoid bodies formed that when adhered and differentiated formed high numbers of TH+ cells. If F12 was omitted very few TH+ cells were observed.

2. Neurospheres

Neurospheres contained neuronal cells (NF200+ve). Neurospheres also included glial cells (GFAP+ve) and oligodendrocytes.

### EXAMPLE 6

Example 5 was repeated utilising human ES cells and a minimal media consisting of DMEM and 100 μM L-proline as outlined in the schematic of FIG. 12. Results were similar to those described in Example 5.

Human ES cell culture, embryoid body /cell aggregate formation, adherent culture and passaging to form neurospheres or cell reaggregates were essentially conducted as outlined in Example 4.

L-Proline

EB¹⁷ bodies formed in medium that contained DMEM and 100 μM L-proline were comprised of proliferating cells Ki67+ve), neuronal cells (NF200+ve), and a high proportion (˜50%) TH+ve cells. When the medium excluded L-proline the TH+ve cell content of EB¹⁷ bodies was reduced significantly. Generation of EBs with high proportions of TH+ cells occurred in the absence of FGF2. Cells grown in the DMEM and N2 or ITSS did not produce a significant population of TH+ cells (see FIG. 13F.).

Formation of neurospheres was achieved as described in Example 4. The neurospheres formed were seeded onto poly-L-ornithine/laminin coated plates and allowed to adhere and differentiate. Optionally in this culture stage, neurospheres can be maintained in media containing combinations of RA, 50% MEDII and L-Proline. In an alternative treatment, during stage A, the embryoid bodies (EB⁹) can be seeded onto poly-L-ornithine/laminin coated culture plates and cultured for 6 to 8 days to permit neuronal differentiation.

In an alternative treatment, embryoid bodies cultured from stage A can be triturated and resuspended in a minimal media (DMEM/F12 and N2 or ITSS). Optionally this media can also include combinations of FGF, MEDII, RA and L-Proline. The aggregates formed can also be seeded onto poly-L-Ornithine/laminin coated plates and allowed to adhere and differentiate.

Results of these experiments are shown in FIG. 14.

Results

1. L-proline

In the presence of L-Proline embryoid bodies formed that when adhered and differentiated formed high numbers of TH+cells. If F12 was omitted very few TH+ cells were observed.

2. Neurospheres

Neurospheres contained neuronal cells (NF200+ve). Neurospheres also included glial cells (GFAP+ve) and oligodendrocytes.

### EXAMPLE 7

Example 4 was repeated with modifications illustrated in FIG. 15. Single Human ES cells (trypsinised) were grown in a standard suspension culture containing 50% MEDII filtrate in the presence of FGF2. At day 9 the embryoid bodies so formed (EBM9) were transferred to poly-n-ornithine/laminin coated plates in the same serum-free MEDII filtrate culture medium, maintained for a further 8 days and allowed to adhere. The embryoid bodies so formed (EBM 17) were then trypsinised to near single cell form. A cell suspension of 100,000 cells/μl was stereotaxically injected (100,000 cells/μl per animal) into the 6-OHDA lesioned striatum of eight Sprague-Dawley rats. A group of 5 Rats was also included that did not receive cell implants and acted as sham controls. Rats were maintained under conditions of immunosuppression using Cyclosporin A (10 mg/kg) for a period of 8 weeks and rotational data was collected. Grafted human cells were detected using a human Alu-repeat DNA detection system. After the 8 week period the 8 implanted Rats showed a statistically significant reduction in their rotational scores compared to the control group. (Single Factor ANOVA, p=0.047) (data not shown). Immunohistochemical characterisation of the human cell implants revealed neural lineages and low numbers of neural cells positive for the dopaminergic neurone marker, Tyrosine Hydroxylase (TH+).

Results from this experiment are presented in FIG. 16 and depict the neural differentiation of the implanted cells after an 8 week period.

Results

1. MEDII Filtrate

Serum Free MEDII filtrate contains F12 medium, which includes L-Proline (75 μM final concentration in conditioning media).

2. FGF2

FGF2 was included in the culture medium to prepare cells for transplantation. However inclusion of FGF2 is optional.

3. Adherent Culture

EBM⁹s are cultured on laminim/polyornithine coated plates for a period of up to 8 days to form EB¹⁷s.

4. Implant Differentiation

Implanted cells differentiated to form neurones (neurones (TH+), glial cells (GFAP positive),

### EXAMPLE 8

Example 7 was repeated utilising minimal culture media (DMEM:F12, and ITSS or N2) with or without 10 μg/ml FGF2 in both stages A and B. This produced embryoid bodies at days 15 to 17 (EB 15 to 17) containing high numbers of TH positive neuronal cells (see Example 5).

The cells were trypsinised to near single cell suspension and transplanted in 1 μl (100,000 cells) into a rat model as described above.

1. FGF2 was not included in the culture medium to prepare cells for transplant. However inclusion of FGF2 in the culture medium is optional.

2. Adherent culture

EB⁹s are cultured on laminin/polylornithine coated plates a further period of up to 8 days.

It will be understood that the invention disclosed and defined in this specification extends to all alternative combinations of two or more of the individual features mentioned or evident from the text or drawings. All of these different combinations constitute various alternative aspects of the invention.

It will also be understood that the term “comprises” (or its grammatical variants) as used in this specification is equivalent to the term “includes” and should not be taken as excluding the presence of other elements or features.

Documents included in this specification are for reference purposes and their inclusion is not an admission that such documents form part of the common general knowledge in the relevant art.

### REFERENCES

Anderson D J (1989). The neural crest lineage problem: neuropoiesis. Neuron 3,1-12.
Bain G, Kitchens D, Yao M, Huettneer J E, Gottlieb D I (1995). Embryonic stem cells express neural properties in vitro. Dev Biol 168, 342-357.
Bjornson C R, Rietze R L, Reynolds B A, Magli M C, Vescovi A L (1999). Turning brain into blood: a hematopoietic fate adopted by adult neural stem cells in vivo. Science 283, 534-537.
Li M, Pevny L, Lovell-Badge R, Smith A (1998). Generation of neural precursors from embryonic stem cells by lineage selection. Current Biol 8, 971-974.
Okabe S, Forsberg-Nilsson K, Spiro A C, Segal M, McKay R D (1996). Development of neuronal precursor cells and functional postmitotic neurons from embryonic stem cells in vitro. Mech Dev 59, 89-102.
Renoncourt Y, Carrol P, Filippi P, Aru V, Alonso S (1998). Neurons derived in vitro from ES cells express homeoproteins characteristic of motorneurons and intemeurons. Mech Dev 79, 185-197.
Tropepe V, Hitoshi S, Sirard C, Mak T W, Rossant J, van der Kooy D (2001). Direct neural fate specification from embryonic stem cells: a primitive mammalian neural stem cell stage acquired through a default mechanism. Neuron 30, 65-78

1. A method of producing neural progenitor cells and/or neuronal cells which method includes
providing
a source of pluripotent cells;
a cell aggregate-inducing culture medium; and
a neural inducing supplement;
culturing the pluripotent cells in the cell aggregate-inducing culture medium, in the presence of the neural inducing supplement, for a period sufficient to permit cell aggregates or embryoid bodies (EB's) to form, wherein the EB's include neural progenitor cells; and
culturing the cell aggregates including neural progenitor cells for a period sufficient to permit neuronal differentiation.

2. A method according to claim 1 wherein the cell aggregate-inducing medium is a Dulbecco's Modified Eagles Medium (DMEM).

3. A method according to claim 1 wherein the neural inducing supplement is selected from the group consisting of ITSS, B27 and N2.

4. A method according to claim 1 wherein the cell aggregate-inducing medium is serum-free.

5. A method according to claim 1 wherein the cell aggregation is conducted in the absence of a fibroblast growth factor.

6. A method according to claim 1 wherein the cell aggregate-inducing medium includes a fibroblast growth factor.

7. A method according to claim 6 wherein the fibroblast growth factor is FGF-2.

8. A method according to claim 1 wherein the cell aggregate-inducing medium further includes retinoic acid, an isomer thereof, precursor thereof or derivative thereof.

9. A method according to claim 1 wherein the initial culturing step continues for approximately 3 to 10 days.

10. A method according to claim 9 wherein the initial culturing step continues for approximately 9 days.

11. A method according to claim 9 wherein the cell aggregates are cultured for an additional approximately 4 to 20 days to permit neuronal differentiation.

12. A method according to claim 11 wherein the cell aggregates are cultured for an additional approximately 6 to 9 days.

13. A method according to claim 1 wherein the neuronal differentiation is conducted in a medium that is the same as the cell aggregate-inducing medium?.

14. A method according to claim 13 wherein the medium includes neural inducing supplement.

15. A method according to claim 1 wherein the cell aggregation is conducted in suspension culture and the neuronal differentiation is conducted in suspension or adhesion culture.

16. A method according to claim 15 wherein the neuronal differentiation is conducted in adhesion culture.

17. A method according to claim 1 wherein the pluripotent cells include or are derived from one or more of the group consisting of embryonic stem (ES) cells, early primitive ectoderm-like (EPL) cells in vivo or in vitro derived ICM/epiblast, in vivo or in vitro derived primitive ectoderm, primordial germ cells (EG cells), teratocarcinoma cells (EC cells), and pluripotent cells derived by dedifferentiation or by nuclear transfer.

18. A method according to claim 17 wherein the pluripotent cells are ES cells.

19. A method according to claim 18 wherein the ES cells are selected for SSEA4 expression, and are expanded in vitro.

20. A method according to claim 17 wherein the pluripotent cells are mammalian ES or EPL cells.

21. A method according to claim 20 wherein the pluripotent cells are human ES or EPL cells.

22. A method according to claim 17 wherein the pluripotent cells are subjected to a cell separation step prior to culturing.

23. A method according to claim 22 wherein the cells are treated with trypsin prior to the initial culturing step.

24. A method of producing tyrosine hydroxylase positive cells which method includes
providing
a source of pluripotent cells;
a cell aggregate-inducing culture medium;
a neural inducing supplement; and
a tyrosine hydroxylase (TH)-inducing supplement;
culturing the pluripotent cells in the cell aggregate-inducing culture medium, in the presence of the neural inducing supplement and TH-inducing supplement, for a period sufficient to permit cell aggregates or embryoid bodies (EB's) to form, wherein the EB's include neural progenitor cells; and
culturing the cell aggregates including neural progenitor cells for a period sufficient to permit differentiation of neuronal cells, wherein said neuronal cells express tyrosine hydroxylase.

25. A method according to claim 24 wherein the cell aggregate-inducing medium is a Dulbecco's Modified Eagles Medium (DMEM).

26. A method according to claim 24 wherein the neural inducing supplement is selected from the group consisting of ITSS, B27 and N2.

27. A method according to claim 24 wherein the TH-inducing supplement includes a source of proline.

28. A method according to claim 27 wherein the concentration of proline in the TH-inducing supplement is approximately 50 μM or greater.

29. A method according to claim 24 wherein the TH-inducing supplement is Ham's F12 nutrient media.

30. A method according to claim 24 wherein the TH-inducing supplement is a MED II conditioned medium or filtrate thereof.

31. A method according to claim 24 wherein the cell aggregate-inducing medium is serum-free.

32. A method according to claim 24 wherein the cell aggregation is conducted in the absence of a fibroblast growth factor.

33. A method according to claim 24 wherein the cell aggregate-inducing medium includes a fibroblast growth factor.

34. A method according to claim 33 wherein the fibroblast growth factor is FGF-2.

35. A method according to claim 24 wherein the cell aggregate-inducing medium further includes retinoic acid, an isomer thereof, precursor thereof or derivative thereof.

36. A method according to claim 24 wherein the initial culturing step continues for approximately 3 to 10 days.

37. A method according to claim 36 wherein the initial culturing step continues for approximately 9 days.

38. A method according to claim 36 wherein the cell aggregates are cultured for an additional approximately 4 to 20 days to permit neuronal differentiation.

39. A method according to claim 38 wherein the cell aggregates are cultured for an additional approximately 6 to 9 days.

40. A method according to claim 24 wherein the neuronal differentiation is conducted in a medium that is the same as the cell aggregate-inducing medium.

41. A method according to claim 40 wherein the medium includes neural inducing supplement and TH-inducing supplement.

42. A method according to claim 24 wherein the cell aggregation is conducted in suspension culture and the neuronal differentiation is conducted in suspension or adhesion culture.

43. A method according to claim 42 wherein the neuronal differentiation is conducted in adhesion culture.

44. A method according to claim 24 wherein the cell aggregates so formed include at least approximately 5% neuronal cells.

45. A method according to claim 44 wherein the cell aggregates so formed include approximately 50% neuronal cells.

46. A method according to claim 44 wherein the cell aggregates so formed include at least approximately 5% tyrosine hydroxylase positive (TH+ve) cells.

47. A method according to claim 46 wherein the cell aggregates so formed include approximately 50% tyrosine hydroxylase positive (TH+ve) cells.

48. A method of producing neurospheres, which method includes
providing
a source of pluripotent cells;
a cell aggregate-inducing culture medium;
a neural inducing supplement;
optionally a TH-inducing supplement; and
a neurosphere-inducing culture medium;
culturing the pluripotent cells in the cell aggregate-inducing culture medium, in the presence of the neural inducing supplement and optionally in the presence of the TH-inducing supplement for a period sufficient to permit cell aggregates or embryoid bodies (EB's) to form;
disaggregating the embryoid bodies;
culturing the cells so released in the neurosphere-inducing culture medium to form neurospheres; and
harvesting the neurospheres so formed.

49. A method according to claim 48 wherein the cell aggregate-inducing medium is a Dulbecco's Modified Eagles Medium (DMEM).

50. A method according to claim 48 wherein the neural inducing supplement is selected from the group consisting of ITSS, B27 and N2.

51. A method according to claim 48 wherein the TH-inducing supplement is present and includes a source of proline.

52. A method according to claim 51 wherein the concentration of proline in the TH-inducing supplement is approximately 50 μM or greater.

53. A method according to claim 48 wherein the TH-inducing supplement is Ham's F12 nutrient media.

54. A method according to claim 48 wherein the TH-inducing supplement is a MED II conditioned medium or filtrate thereof.

55. A method according to claim 48 wherein the cell aggregate-inducing medium is serum-free.

56. A method according to claim 48 wherein the cell aggregation is conducted in the absence of a fibroblast growth factor.

57. A method according to claim 48 wherein the neurosphere-inducing culture medium includes a serum-free medium supplemented with a source of proline.

58. A method according to claim 48 wherein the neurosphere-inducing culture medium includes a serum-free Dulbecco's Modified Eagles Medium (DMEM) supplemented with Ham's F12 nutrient media and/or a MED II conditioned medium or filtrate thereof.

59. A method according to claim 48 wherein the neurosphere-inducing culture medium further includes a growth factor from the FGF family, optionally in the presence of additional growth factors and/or differentiation agents.

60. A method according to claim 59 wherein the culture medium further includes FGF-2 optionally together with one or more of heparin, B27 and ITSS.

61. A method according to claim 48 wherein the neurosphere induction continues for approximately 3 to 9 days.

62. A method according to claim 48 wherein the initial culturing step continues for approximately 6 to 25 days.

63. A method according to claim 48 which method further includes maintaining the neurospheres in a serum-free culture media prior to harvesting.

64. A method according to claim 63 wherein the neurospheres are maintained in media for approximately 1 to 21 days.

65. Neurospheres produced by a method according to claim 48 or the partially or terminally differentiated progeny thereof.

66. Neurospheres according to claim 65 characterised in that they exhibit a reduced propensity to generate teratomas in vivo or the partially or terminally differentiated progeny thereof.

67. Neurospheres according to claim 65 wherein the neurospheres include proliferating cells including neuronal cells, a proportion of which are dopaminergic.

68. Neurospheres according to claim 65 wherein the neurospheres include proliferating cells including neural progenitors, neuronal progenitors and glial progenitors.

69. Neurospheres according to claim 65 further including glial cells.

70. A method of producing neuronal and/or neural progenitor cells which method includes
providing
a source of neurospheres; and
a neuronal differentiation culture medium; and
culturing the neurospheres in the presence of the neural differentiation medium for a period sufficient to permit neuronal differentiation.

71. A method according to claim 70 wherein the neurospheres are produced according to the method of claim 48.

72. A method according to claim 71 wherein the neuronal differentiation medium is a Dulbecco's Modified Eagles Medium (DMEM) supplemented with a source of proline or Hams F12 or a MED II conditioned medium or filtrate thereof.

73. A method according to claim 72 wherein the neurospheres are cultured in the absence of a fibroblast growth factor.

74. Differentiated neuronal cells produced by a method according to claim 70.

75. Differentiated neuronal cells according to claim 74 wherein approximately 5% to approximately 50% of the cells are neuronal cells.

76. Differentiated neuronal cells according to claim 74 wherein approximately 5% to approximately 50% of the cells are tyrosine hydroxylase positive (TH+ve).

77. Neuronal cells and/or neural progenitor cells whenever produced by a method according to claim 1.

78. Tyrosine hydroxylase positive cells whenever produced by a method according to claim 24.

79. Use of neurospheres, neuronal or neural progenitor cells or tyrosine hydroxylase positive cells according to any one of claims 65, 74, 77 or 78 or their differentiated or partially differentiated progeny in human cell therapy or transgenic animal production.

80. Use of neurospheres, neuronal or neural progenitor cells or tyrosine hydroxylase positive cells according to any one of claims 65, 74, 77 or 78 or their differentiated or partially differentiated progeny in human or animal gene therapy.

81. A method for the treatment of neuronal and related diseases, which method includes treating a patient requiring such treatment with genetically modified or unmodified neurospheres, neuronal or neural progenitor cells or tyrosine hydroxylase positive cells according to any one of claims 65, 74, 77 or 78, or their partially differentiated or terminally differentiated progeny, through human or animal cell or gene therapy.

82. A method according to claim 81 wherein the disease to be treated is Parkinson's disease or related diseases.

83. Use of neurospheres, neuronal or neural progenitor cells or tyrosine hydroxylase positive cells according to any one of claims 65, 74, 77 or 78, or their differentiated or partially differentiated progeny, for the preparation of a medicament for treatment of neuronal and related diseases.

84. Use according to claim 83 wherein the disease to be treated is Parkinson's disease or related diseases.
